SEQUENCE LISTING (7 sequences)

SEQ ID NO: 1 - DNA, 864 bp, Artificial
  Synthetic
ctcgagtaaactttactgtcgttttgttagctaaaaggaaaaaattatgggcttcctggaccgtctgggccgtgtcgtgaaagcgaacctgaatgatatggtgtcgaaagcagaagacccggaaaaaatcctggaacaggcagtcgctgatatgggcgaaagcctggtccaactgcgtcagtctgtggcgcgtgcgattgcggcccagaagaaaaccgaacagcaactgatcaaaaaccaaaccgaagcgaccacgtggcagaagaaagcggaactggccattaaaaatggtcgtgaagatctggcacgcgaagctctggttcgtaagaaaacctttgcagacacggcagctgtcctgcagcaacagctgacgcaacagaacgcccaagttaaaaccctgaaagaaaatctgctggcactggaaagtaaaatccaggaagctaaaaccaagaaagatatgctgaaagcacgcgctaacgcggccaaagcgaatgcccaactgcagagtacgatgaacaatattgataccagctctgcgatgtccgcctttgaacgtatggaagacaaaatcatggaactggaagcacagtctgaagctaccaacgtgctgggcagtgcgtccctggatcaagaattcgcgcagctggaagcctcaaattcgattgatgacgaactggccatgctgaaagcagcttcacaggaagcaccggctctggaagcggccaaaatggttgatgaaatggaagaagtgtcggacaccccggttgaaacgccggaaccgaccgacgcggactttgcggctgttgatgcggaactggaagcactgcgtacccagatgaaaaacctgtaaaagcttggcgcgcc

SEQ ID NO: 2 - DNA, 1083 bp, Artificial
  Synthetic
catatgctgacggcagaacgtatcaagtttacccagaagcgtggctttcgtcgtgtcctgaaccaacgtgtggatgcgtattttgctgaacatggtctgacccagcgtgataacccgtcaatgtatctgaaaacgctgattatcgtcctgtggctgttctcggcgtgggcctttgtgctgttcgcaccggttatttttccggtccgcctgctgggttgcatggttctggcaatcgctctggcggccttttcattcaatgtcggccatgatgcaaaccacaatgcttatagctctaacccgcatattaatcgtgttctgggcatgacctacgacttcgtcggtctgagttcctttctgtggcgttatcgccacaactacctgcatcacacctatacgaatattctgggccatgatgtggaaatccacggcgacggtgccgttcgtatgagcccggaacaggaacatgtgggtatctaccgctttcagcaattctatatttggggcctgtacctgtttatcccgttctattggtttctgtatgatgtctacctggtgctgaacaaaggtaagtaccatgaccacaaaattccgccgtttcaaccgctggaactggcatctctgctgggcatcaagctgctgtggctgggctatgtgttcggtctgccgctggcgctgggcttttcaattccggaagtcctgatcggtgcctcggtgacctatatgacgtacggcattgtggtttgtaccatcttcatgctggctcatgttctggaaagcaccgaatttctgacgccggatggcgaatctggtgcaattgatgacgaatgggctatttgccagatccgtaccacggcgaacttcgccaccaacaatccgttctggaactggttttgtggcggtctgaatcaccaagtgacgcatcacctgtttccgaacatttgccatatccactatccgcagctggaaaacatcatcaaggacgtttgtcaagaattcggtgtggaatataaagtttacccgacctttaaggcagctattgcgagtaattaccgctggctggaagcgatgggcaaggcctcctaa

SEQ ID NO: 3 - DNA, 1053 bp, Artificial
  Synthetic
atgcaatccaccgttcgttctccgggtagccgtgaaagcctgcgccaagacctgccgtttacgctgaaagacgtgaaagccgccattccggattattgcttccagccgagcgtgtttcgttctctggcgtactttttcctggatatcggtattatcgcaggcctgtatgctattgcggcctacctggactcttggtttttctatccgatcttttggttcgcacagggtacgatgttttgggctctgttcgtggttggccatgattgtggccacggtagcttctctcgcagtaaatttctgaacgacctgatcggtcatctgagccacaccccgattctggttccgtttcatggctggcgtatttcacaccgcacccatcactcgaacacgggtaatatcgataccgacgaaagctggtatccgattccggaatctaaatacgatcagatgggcttcgcggaaaaactggtccgtttttatgcgccgctgattgcctatccgatctacctgtttaaacgcagtccgggtcgtggtccgggttcccatttctcaccgaaatcgccgctgtttaaaccggcggaacgcaatgatattatcctgtccacggcagctattatcgccatggtcggttttctgggctggttcaccgtgcaatttggtctgctggccttcgttaaattttatttcgtcccgtacgtgatttttgttatctggctggatctggttacgtatctgcatcacaccgaagcagacatcccgtggtaccgtggtgatgactggtattacctgaaaggcgctctgagtaccattgatcgcgactatggtattttcaacgaaatccatcacaatattggcacgcatgtcgcccatcacatctttcacaccattccgcattaccacctgaaagatgcgaccgaagccatcaaaccgctgctgggcgactattaccgtgtgagccatgcaccgatttggcgtagctttttccgctcccagaaagcgtgtcactacatcgccgaccaaggcagccacctgtattatcaaccgaaaaaataa

SEQ ID NO: 4 - DNA, 1121 bp, Artificial
  Synthetic
atgctgacggcagaacgtatcaagtttacccagaagcgtggctttcgtcgtgtcctgaaccaacgtgtggatgcgtattttgctgaacatggtctgacccagcgtgataacccgtcaatgtatctgaaaacgctgattatcgtcctgtggctgttctcggcgtgggcctttgtgctgttcgcaccggttatttttccggtccgcctgctgggttgcatggttctggcaatcgctctggcggccttttcattcaatgtcggccatgatgcaaaccacaatgcttatagctctaacccgcatattaatcgtgttctgggcatgacctacgacttcgtcggtctgagttcctttctgtggcgttatcgccacaactacctgcatcacacctatacgaatattctgggccatgatgtggaaatccacggcgacggtgccgttcgtatgagcccggaacaggaacatgtgggtatctaccgctttcagcaattctatatttggggcctgtacctgtttatcccgttctattggtttctgtatgatgtctacctggtgctgaacaaaggtaagtaccatgaccacaaaattccgccgtttcaaccgctggaactggcatctctgctgggcatcaagctgctgtggctgggctatgtgttcggtctgccgctggcgctgggcttttcaattccggaagtcctgatcggtgcctcggtgacctatatgacgtacggcattgtggtttgtaccatcttcatgctggctcatgttctggaaagcaccgaatttctgacgccggatggcgaatctggtgcaattgatgacgaatgggctatttgccagatccgtaccacggcgaacttcgccaccaacaatccgttctggaactggttttgtggcggtctgaatcaccaagtgacgcatcacctgtttccgaacatttgccatatccactatccgcagctggaaaacatcatcaaggacgtttgtcaagaattcggtgtggaatataaagtttacccgacctttaaggcagctattgcgagtaattaccgctggctggaagcgatgggcaaggcctcctaaagctcgagtaaactttactgtcgttttgttagctaaaagga

SEQ ID NO: 5 - protein, 267 aa, Artificial
  Synthetic
MGFLDRLGRVVKANLNDMVSKAEDPEKILEQAVADMGESLVQLRQSVARAIAAQKKTEQQLIKNQTEATTWQKKAELAIKNGREDLAREALVRKKTFADTAAVLQQQLTQQNAQVKTLKENLLALESKIQEAKTKKDMLKARANAAKANAQLQSTMNNIDTSSAMSAFERMEDKIMELEAQSEATNVLGSASLDQEFAQLEASNSIDDELAMLKAASQEAPALEAAKMVDEMEEVSDTPVETPEPTDADFAAVDAELEALRTQMKNL

SEQ ID NO: 6 - protein, 359 aa, Artificial
  Synthetic
MLTAERIKFTQKRGFRRVLNQRVDAYFAEHGLTQRDNPSMYLKTLIIVLWLFSAWAFVLFAPVIFPVRLLGCMVLAIALAAFSFNVGHDANHNAYSSNPHINRVLGMTYDFVGLSSFLWRYRHNYLHHTYTNILGHDVEIHGDGAVRMSPEQEHVGIYRFQQFYIWGLYLFIPFYWFLYDVYLVLNKGKYHDHKIPPFQPLELASLLGIKLLWLGYVFGLPLALGFSIPEVLIGASVTYMTYGIVVCTIFMLAHVLESTEFLTPDGESGAIDDEWAICQIRTTANFATNNPFWNWFCGGLNHQVTHHLFPNICHIHYPQLENIIKDVCQEFGVEYKVYPTFKAAIASNYRWLEAMGKAS

SEQ ID NO: 7 - protein, 350 aa, Artificial
  Synthetic
MQSTVRSPGSRESLRQDLPFTLKDVKAAIPDYCFQPSVFRSLAYFFLDIGIIAGLYAIAAYLDSWFFYPIFWFAQGTMFWALFVVGHDCGHGSFSRSKFLNDLIGHLSHTPILVPFHGWRISHRTHHSNTGNIDTDESWYPIPESKYDQMGFAEKLVRFYAPLIAYPIYLFKRSPGRGPGSHFSPKSPLFKPAERNDIILSTAAIIAMVGFLGWFTVQFGLLAFVKFYFVPYVIFVIWLDLVTYLHHTEADIPWYRGDDWYYLKGALSTIDRDYGIFNEIHHNIGTHVAHHIFHTIPHYHLKDATEAIKPLLGDYYRVSHAPIWRSFFRSQKACHYIADQGSHLYYQPKK

(19) United States
(12) Patent Application Publication
(10) Pub. No.: US 2024/0100168 A1
(43) Pub. Date: Mar. 28, 2024

(54) LIPID COMPOSITIONS AND METHODS OF PREPARATION THEREOF

(71) Applicant: Wake Forest University, Winston-Salem, NC (US)

(72) Inventors: Floyd H. Chilton, III, Oro Valley, AZ (US); Leslie B. Poole, Winston-Salem, NC (US)

(21) Appl. No.: 17/996,311

(22) PCT Filed: Apr. 16, 2021
(86) PCT No.: PCT/US21/27716

§ 371 Date: Oct. 14, 2022

Related U.S. Application Data
(60) Provisional application No. 63/011,680, filed on Apr. 17, 2020.

(51) Int. Cl. A61K 47/54; A23K 20/158; A23K 50/80; A61K 31/202; C12N 9/02; C12P 7/64; C12P 7/6481

(52) U.S. Cl. CPC A61K 47/549; A23K 20/158; A23K 50/80; A61K 31/202; A61K 47/545; A61K 47/548; C12N 9/0071; C12P 7/64; C12P 7/6481; C12Y 114/19

(57) ABSTRACT

This disclosure demonstrated that cyanobacteria bioengineered with cyanobacterial lipid-biosynthetic-promoting genes could produce large quantities of SDA, as well as rarely observed ETA. Importantly, the biosynthesized omega-3 fatty acids, such as SDA and ETA, are found conjugated to more bioavailable glycolipids, including MGDG and DGDG. Novel compositions include MGDG, DGDG, SQDG, and PG molecular species that contain the following n-3 PUFAs and n-3 LC-PUFAs at both sn-1 and sn-2 positions: 18:3/18:4, 18:3/20:4, 18:3/20:5, 18:4/18:4, 18:4/20:4, 18:4/20:5. These compositions are not found in nature and result from the engineering of cyanobacteria; and can serve as highly bioavailable, cost-effective anti-inflammatory compounds. These compositions, therefore, have strong anti-inflammatory properties with the likely capacity to block activities of cyclooxygenases, lipoxygenases, and cytochrome P450s that metabolize PUFAs and LC-PUFAs to pro-inflammatory mediators. These compounds also inhibit the uptake of pro-inflammatory PUFAs and LC-PUFAs into cells and especially inflammatory cells.

### CROSS-REFERENCE TO RELATED APPLICATIONS

The present application is a National Stage Application under 35 U.S.C. § 371 of International Application No. PCT/US21/27716 filed Apr. 16, 2021, which claims priority under 35 U.S.C. § 119(e) to U.S. Provisional Patent Application No. 63/011,680, filed Apr. 17, 2020. The entire contents of these applications are incorporated herein by reference in their entirety.

### SEQUENCE LISTING

This application contains a Sequence Listing which has been submitted electronically in txt format and is hereby incorporated by reference in its entirety. Said txt copy, created on Oct. 14, 2022, is named “00051_Sequence Listing.txt” and is 14,746 bytes in size.

### FIELD OF THE INVENTION

The disclosure relates generally to lipid compositions and more specifically to novel lipid composition comprising glycolipid-associated omega-3 fatty acids.

### BACKGROUND OF THE INVENTION

Eighteen-carbon (18C), omega-3 (ω-3 or n-3) polyunsaturated fatty acids (PUFAs) and particularly n-3 long-chain (LC, >20 carbons) PUFAs have been shown to exert anti-inflammatory and cardioprotective roles in cardiovascular disease and several inflammatory diseases (1). Additionally, n-3 LC-PUFAs are essential for early childhood development, and deficiencies of n-3 LC-PUFAs are associated with mental disorders and cognitive decline (2-5). Consequently, several health organizations recommend increasing dietary consumption of n-3 PUFAs and LC-PUFAs, resulting in rapidly growing markets for these in functional foods, pharmaceuticals, dietary supplements, and infant formulas (6, 7).

However, expansions in demand for n-3 PUFAs and n-3 LC-PUFAs have raised vital questions about their sustainability. For example, fish represent the predominant source of n-3 LC-PUFAs; however, wild-caught fish are at or beyond exploitable limits, and more than half of fish consumed are farmed (7). Krill oil, as another unsustainable source of n-3 LC PUFAs, exerts even greater strains on the global health of ocean fisheries. Approximately 75% of the global supply of n-3 LC-PUFAs is currently utilized by aquaculture, which has led to a shift to potentially pro-inflammatory n-6 PUFA-based vegetable (such as soybean and rapeseed) oil products, decreasing the nutritional quality of the farmed fish (8-11). Furthermore, there is a growing need for dietary n-3 PUFAs and n-3 LC-PUFAs in terrestrial livestock to enrich levels in meat, milk, and egg products (12, 13).

Potential solutions to the growing demand for n-3 18C-PUFAs and LC-PUFAs are plant and algae-based sources produced through solar energy-dependent processes (7). Most plant-sourced, n-3 PUFA-containing oils, such as flaxseed oil, are enriched with the 18C-PUFA α-linolenic acid (ALA; 18:4, n-3), which has the potential to be converted to n-3 LC-PUFAs such as eicosapentaenoic acid (EPA, 20:5) and docosahexaenoic acid (DHA, 22:6). However, humans and most animals, including cold-water species of fish, are inefficient at converting ALA into EPA and DHA. The rate-limiting steps in this conversion are the desaturation steps, and particularly the Δ6 desaturase (FIG. 1A). However, the product of Δ6 desaturase, stearidonic acid (SDA, 18:4 n-3), bypasses this rate-limiting step; several human and animal studies show that seed oils containing SDA are more efficiently converted to EPA than those with ALA (14-16). SDA-containing seed oils from relatively rare plant species have been commercialized, and common plant seed oils such as soybeans and canola have been genetically engineered to have enriched content of SDA (20-29% of total fatty acids) (Ursin V M. J Nutr. 2003; 133:4271-4; Eckert H, et al. Planta. 2006; 224:1050-7.). Human clinical studies show that SDA-enhanced soybean oil significantly elevates n-3 LC-PUFAs and improves markers of cardiovascular health (17, 18). However, the feasibility of these commercial applications and the stability of these transgenic plants remain to be determined. More recently, there has been a marked increase in the production and sales in human consumer markets of n-3 LC-PUFAs (EPA and DHA) from phototrophic algae. Nevertheless, there are significant production cost barriers in supplying highly-bioavailable forms to the rapidly expanding aquaculture and livestock feed markets as well as human consumption. Additionally, novel compositional forms of n-3 PUFAs and LC-PUFAs are needed that have potent anti-inflammatory effects.

Accordingly, there exists a need for novel highly bioavailable forms of n-3 PUFAs and n-3 LC-PUFAs at a low cost. There is also a demand for new compositions of n-3 PUFAs and n-3 LC-PUFAs that can serve as novel anti-inflammatory compounds directed at a variety of inflammatory diseases including but not limited to cardiovascular disease, diabetes, asthma, allergies, cancer, and Alzheimer's disease.

### SUMMARY OF THE INVENTION

This disclosure addresses the need mentioned above in a number of aspects. In one aspect, this disclosure provides never-described, novel lipid compositions having a lipid profile comprising lipids and an omega-3 fatty acid, such as α-linolenic acid (ALA, 18:3 n-3), stearidonic acid (SDA, 18:4, n-3), omega-3 arachidonic acid (omega-3 ETA, 20:4, n-3), eicosapentaenoic acid (EPA, 20:5, n-3), wherein the lipids comprise a glycolipid and a fraction of the omega-3 fatty acid is conjugated to at least one of the sn-1 and sn-2 positions of a glycolipid head group, such as monogalactosyldiacylglycerol (MGDG), digalactosyldiacylglycerol (DGDG), and sulfoquinovosyldiacylglycerol (SQDG). In some embodiments, the lipid composition further comprises two omega-3 fatty acid chains, with these chains conjugated to both the sn-1 and sn-2 positions of the MGDG, DGDG, or SQDG.

In some embodiments, the lipids further comprise a phosphoglycerolipid. In some embodiments, the lipid composition further comprises two omega-3 fatty acid chains, with these chains conjugated to both the sn-1 and sn-2 positions of the phosphatidylglycerol (PG).

In some embodiments, the omega-3 fatty acid is conjugated to at least one of the sn-1 and sn-2 positions, and in many cases at both the sn-1 and sn-2 positions of MGDG, DGDG, SGQG, or PG. In some embodiments, the novel compositions include MGDG, DGDG, SQDG, and PG molecular species that contain the following n-3 PUFAs and n-3 LC-PUFAs at their sn-1 and sn-2 positions: 16:3/18:4, 17:2/18:4, 17:3/18:4, 18:2/20:4, 18:3/20:3, 18:3/18:4, 18:3/20:4, 18:3/20:5, 18:4/18:4, 18:4/20:4, 18:4/20:5, 20:4/20:5, and other novel previously undescribed molecular species as shown in Tables 2 and 3.

In some embodiments, the novel compositions that include MGDG, DGDG, SQDG, and PG molecular species that contain the following n-3 PUFAs and n-3 LC-PUFAs at their sn-1 and sn-2 positions: 16:3/18:4, 17:2/18:4, 17:3/18:4, 18:2/20:4, 18:3/20:3, 18:3/18:4, 18:3/20:4, 18:3/20:5, 18:4/18:4, 18:4/20:4, 18:4/20:5, 20:4/20:5 are highly-bioavailable sources of n-3 PUFAs and n-3 LC-PUFAs that enrich circulating, cellular and tissue levels in animals and humans.

In some embodiments, the novel compositions that include MGDG, DGDG, SQDG, and PG molecular species that contain the following n-3 PUFAs and n-3 LC-PUFAs at their sn-1 and sn-2 positions: 16:3/18:4, 17:2/18:4, 17:3/18:4, 18:2/20:4, 18:3/20:3, 18:3/18:4, 18:3/20:4, 18:3/20:5, 18:4/18:4, 18:4/20:4, 18:4/20:5, 20:4/20:5 have anti-inflammatory properties that block activities of numerous enzymes, such as cyclooxygenases, lipoxygenases, P450s that metabolize PUFAs and LC-PUFAs to pro-inflammatory mediators.

In some embodiments, the novel compositions that include MGDG, DGDG, SQDG, and PG molecular species that contain the following n-3 PUFAs and n-3 LC-PUFAs at their sn-1 and sn-2 positions: 16:3/18:4, 17:2/18:4, 17:3/18:4, 18:2/20:4, 18:3/20:3, 18:3/18:4, 18:3/20:4, 18:3/20:5, 18:4/18:4, 18:4/20:4, 18:4/20:5, 20:4/20:5 inhibit the uptake of pro-inflammatory PUFAs and LC-PUFAs into cells and especially inflammatory cells.

In some embodiments, the lipid composition of any one of the preceding claims, wherein at least 50% of the SDA, the omega-3 ETA, the EPA or the ALA is in the fraction of the omega-3 fatty acid conjugated to the MGDG, the DGDG, or both. In some embodiments, at least 60% of the SDA is in the fraction of the omega-3 fatty acid conjugated to the MGDG, the DGDG, or both. In some embodiments, at least 60% of the omega-3 ETA is in the fraction of the omega-3 fatty acid conjugated to the MGDG, the DGDG, or both. In some embodiments, the lipid composition comprises at least about 10% SDA, at least about 1% omega-3 ETA, at least about 0.1% omega-3 EPA, or at least about 20% ALA, by weight of total fatty acid content. In some embodiments, a total amount of the SDA, the omega-3 ETA, EPA, and the ALA is at least about 20% by weight of total fatty acid content. In some embodiments, the lipid composition comprises between about 5% and about 40% ALA; between about 10% and about 30% SDA; and between about 1% and about 10% omega-3 ETA.

In some embodiments, the lipid composition is produced from a modified cyanobacterium. In some embodiments, the modified cyanobacterium is a species of Anabaena, Leptolyngbya, Lyngbya, Nostoc, Phormidium, Spirulina, Synechococcus, or Synechocystis. In some embodiments, the modified cyanobacterium is Anabaena sp. PCC7120, Synechococcus sp. PCC7002, or Leptolyngbya sp. strain BL0902.

In some embodiments, the lipid compositions and unique molecular species described above is prepared in an administrable form selected from the group consisting of a pharmaceutical formulation, a nutritional formulation, a feed formulation, a dietary supplement, a medical food, a functional food, a beverage product, and a combination thereof.

In another aspect, this disclosure also provides a feed for use in aquaculture comprising the lipid composition described above. Also within the scope of this disclosure is a food or drink additive comprising the lipid composition.

In another aspect, this disclosure further provides a method of producing the lipid composition, as described above. The method comprises (a) culturing a modified microorganism comprising at least one exogenous gene encoding a desaturase in a culture medium under conditions in which the at least one exogenous gene encoding the desaturase is expressed; and (b) enriching the cultured modified microorganism from the culture medium, wherein the cultured modified microorganism produces a greater amount of the lipid than does a culture comprising a control microorganism identical in all respects except that it does not include the at least one exogenous gene encoding the desaturase.

In some embodiments, the method further comprises extracting the lipids and the omega-3 fatty acid from biomass of the cultured modified microorganism.

In some embodiments, at least one exogenous gene encoding the desaturase comprises a first desaturase gene encoding a Δ6 desaturase and a second desaturase gene encoding a Δ15 desaturase. In some embodiments, the desaturase comprises a polypeptide sequence having at least about 75% identity to SEQ ID NO: 6 or 7 or comprising SEQ ID NO: 6 or 7. In some embodiments, the desaturase is encoded by a nucleic acid sequence having at least about 75% identity to SEQ ID NO: 2 or 3 or comprising SEQ ID NO: 2 or 3.

In some embodiments, the modified microorganism further comprises an exogenous gene encoding thylakoid-promoting protein Vipp1. In some embodiments, the Vipp1 comprises a polypeptide sequence having at least about 75% identity to SEQ ID NO: 5 or comprising SEQ ID NO: 5. In some embodiments, the Vipp1 is encoded by a nucleic acid sequence having at least about 75% identity to SEQ ID NO: 1 or comprising SEQ ID NO: 1.

In yet another aspect, this disclosure additionally provides a method for preventing or treating omega-3 fatty acid deficiency in a subject. The method comprises administering an effective dosage amount of the lipid composition or the pharmaceutical composition, as described above, to the subject in need thereof. In some embodiments, the subject is a mammal (e.g., human). In some embodiments, the subject is a human subject having a cardiovascular or inflammatory disease or condition. In some embodiments, the human subject is in need of rapidly supplementing Omega-3 fatty acids to improve metabolic syndrome, or to benefit from the efficacy of Omega-3 fatty acids in modulating inflammation, prevention of premature birth, myocardial ischemia or infarction, transient local cerebral ischemia or stroke, autoimmunity, and thrombotic diseases, organ transplantation, acute phase response, acute respiratory distress syndrome, inflammatory bowel syndrome, and hypertriglyceridemia. In some embodiments, the administration is an enteral or parental administration.

In another aspect, this disclosure provides a method of inhibiting a cyclooxygenase (e.g., COX-1 and COX-2) or a lipoxygenase in a subject. The method comprises administering an effective amount of the lipid composition of any one of claims 1 to 17 or the pharmaceutical composition of claim 26 to the subject in need thereof.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1, and 1C are a set of diagrams showing the example pathways of 18- and 20-carbon fatty acid synthesis in cyanobacteria, map of the three-gene plasmid (pDBV), and fatty acid contents of all constructs. FIG. 1A shows that introduction of double bonds into stearic acid (18:0) involves a series of acyl-lipid desaturases designated DesC, DesA, DesD, and DesB in cyanobacteria which catalyze desaturation at distinct sites of the carbon chain, ultimately producing stearidonic acid (SDA) if all four desaturase steps occur. Addition of two more carbons by an elongase can then form ETA, the omega-3 isomer of arachidonic acid (omega-6 ARA). The three major omega-3 (or n-3) polyunsaturated fatty acids observed in the engineered cyanobacteria are shown, ALA, SDA, and ETA. The structures shown represent a monogalactosyldiacylglycerol (MGDG) backbone and typical 16:0 saturated fatty acid (palmitic acid) at the sn-2 position in addition to the unsaturated fatty acid at sn-1. FIG. 1B shows that to generate the pDBV and other plasmids, pAM4418-derived expression vectors were generated with synthetic genes designed to express: (i) the Δ6 desaturase (DesD) from Synechocystis sp. PCC 6803, (ii) the methyl-end (o3, or Δ15) desaturase (DesB) from Synechococcus sp. PCC 7002, and/or (iii) the “vesicle-inducing protein in plastids” (Vipp1) from Synechococcus sp. PCC 7002 (FIG. 1). Included in the plasmid vector are aadA, conferring resistance to spectinomycin and streptomycin, as well as trpA and rrnB which block continued transcription. FIG. 1C shows that plasmids with one, two or all three of the inserted cyanobacterial genes were constructed and conjugated into Leptolyngbya sp. strain BL0902, transformants were selected on BG-11 agar plates containing spectinomycin and streptomycin, and cultures were grown at 30° C. in BG-11 media, harvested, and dried for fatty acid analysis of lipid content by fatty acid methyl ester (FAME) analysis (gas chromatography with flame ionization detection, GC-FID). Shown are total saturated (magenta), monounsaturated (dark blue) and polyunsaturated (cyan) fatty acid contents (n=5, 8, 3, 3, 4, 2, 3, 3 from left to right).

FIGS. 2A, 2B, and 2C are the quantitative analysis showing 18- and 20-carbon polyunsaturated fatty acids in wild type and engineered cyanobacteria. PUFA analyses of plasmid-bearing Leptolyngbya sp. strain BL0902, as described in FIG. 1C, are shown as means±standard deviation for wild type (WT) and single-gene constructs (FIG. 2A), or double and triple-gene constructs (FIG. 2B), expressed as the mol percent of total fatty acids (very similar to weight percent values). PUFAs produced by WT and pDBV-bearing species of cyanobacteria, including Leptolyngbya sp. strain BL0902, Synechococcus sp. PCC 7002 and Anabaena sp. PCC 7120 are shown as averages of the mass (mg per g of dry weight)±standard deviation (n=5, 3, 3, 3, 2 and 1 from left to right) (FIG. 2C). Those exhibiting a statistically-significant difference in content compared with WT by a Students t-test are indicated with asterisks (*, p<0.05, **, p<0.01, *** p<0.001).

FIGS. 3A, 3B, and 3C show lipid molecular species analysis by LC-MS/MS to assess distribution of PUFAs in engineered and wild type Leptolyngbya sp. strain BL0902. LC-MS/MS was conducted on lipid extracts dissolved in isopropyl alcohol/methanol (50:50), chromatographed on an Accucore C30 column, and introduced by heated electrospray ionization into a Q Exactive HF Hybrid Quadrupole-Orbitrap Mass Spectrometer with MS scans collected in data-dependent mode, as described in Methods. FIG. 3A shows a Venn diagram of the number of distinct molecular species observed for Leptolyngbya BL0902 without (WT) or with conjugation with the pDBV (n=4 for each). FIGS. 3B and 3C show 18- and 20-carbon PUFAs were observed to be complexed to monogalactosyldiacylglycerol (MGDG), digalactosyldiacylglycerol (DGDG), or phosphatidyl glycerol (PG). Distribution of ALA (18:3 n-3), SDA (18:4 n-3), and ETA (20:4 n-3) among the three glycolipids is shown based on relative abundance by mass spectrometry (ion current), showing the selectivity for the lipid backbone for each PUFA (FIG. 3B). Shown in light blue (WT) and dark blue (engineered with pDBV) are the mean±standard deviation of estimates of mg/g of fatty acids based on (i) normalized peak areas from LC/MS, (ii) fraction of total peak area for each species in a sample, and (iii) known total fatty acid yield for that organism from GC-FID analysis. This treatment assumes that all species exhibit the same ionization efficiency (FIG. 3C). Species across the bottom refer to the two acyl chains associated with MGDG (M), DGDG (D) or PG. Note the shift from fewer to more double bonds upon introduction of the pDBV plasmid. Table 1 (underlined compounds) shows all the individual compositions containing n-3 PUFAs and n-3 LC-PUFAs produce by genetically engineering various cyanobacteria strains.

FIG. 4 shows total fatty acid contents of wild type and engineered cyanobacterial strains. Three cyanobacterial strains, Leptolyngbya sp. strain BL0902, Synechococcus sp. PCC 7002 and Anabaena sp. PCC 7120, were selected for testing given their favorable starting contents of linoleic acid (18:2). Wild type (WT) or engineered (+pDBV) strains were grown, harvested and analyzed for fatty acid content (FAME analysis) as described in FIG. 1. Shown are total saturated (magenta), monounsaturated (dark blue), and polyunsaturated (cyan) fatty acid contents (n=5, 3, 3, 3, 2, and 1 from left to right).

### DETAILED DESCRIPTION OF THE INVENTION

This disclosure provides novel lipid compositions having a lipid profile comprising glycolipids and an omega-3 fatty acid (e.g., ALA, SDA, omega-3 ETA, and EPA), wherein a fraction of the omega-3 fatty acid is conjugated to at least one, or both, of the sn-1 and sn-2 positions of a glycolipid head group, such as MGDG, DGDG, or SQDG. The novel lipid compositions may also include phosphoglycerolipids and a fraction of the omega-3 fatty acid that is conjugated to at least one, or both, of the sn-1 and sn-2 positions of the phosphatidylglycerol (PG).

This disclosure utilized an engineered plasmid encoding one or more cyanobacterial acyl-lipid desaturases (e.g., DesB and DesD, representing Δ15 and Δ6 desaturases) and optionally vesicle-inducing protein (Vipp1) to induce production of stearidonic acid (SDA,18:4 n-3) at high levels in cyanobacteria strains such as Anabaena sp. PCC7120, Synechococcus sp. PCC7002, and Leptolyngbya sp. strain BL0902. Lipidomic analysis revealed that in addition to SDA, the rare anti-inflammatory n-3 LC-PUFA eicosatetraenoic acid (omega-3 ETA, 20:4 n-3) was synthesized in these engineered strains, and ˜99% of SDA and omega-3 ETA was complexed to bioavailable MGDG and DGDG species. Importantly, novel molecular species containing ALA, SDA, ETA, and/or EPA in both acyl positions of MGDG and DGDG were observed, for example, in the engineered Leptolyngbya and Synechococcus strains, suggesting that these could provide a rich source of anti-inflammatory molecules. Importantly, the technology described herein utilizes only solar energy and consumes carbon dioxide, but produces large amounts of nutritionally-important n-3 PUFAs and LC-PUFAs in bioavailable forms. Thus, the disclosed technology could have a major impact on sustainable n-3 PUFA sourcing worldwide.

### A. Lipid Compositions and Profiles

(1) Lipid-Associated Omega-3 Fatty Acids

In one aspect, this disclosure provides novel lipid compositions having a lipid profile comprising lipids and an omega-3 fatty acid, such as ALA, SDA, omega-3 ETA, and/or EPA. The lipids comprise glycolipids, and a fraction of the omega-3 fatty acid is conjugated to the sn-1 or sn-2 position or both of the glycolipid head group, such as MGDG, DGDG, or SQDG. The novel lipid compositions may also include phosphoglycerolipids and a fraction of the omega-3 fatty acid that is conjugated to the sn-1 or sn-2 position or both of the phosphatidylglycerol (PG).

In some embodiments, the omega-3 fatty acid is conjugated to both the sn-1 and sn-2 positions of MGDG, DGDG, SGQG, or PG. In some embodiments, the novel lipid compositions include MGDG, DGDG, SQDG, and PG molecular species that are non-naturally occurring (e.g., not produced by wild-type cyanobacteria strains). For example, the lipid compositions contain the following novel n-3 PUFAs and n-3 LC-PUFAs at their sn-1 and sn-2 positions: 16:3/18:4, 17:2/18:4, 17:3/18:4, 18:2/20:4, 18:3/20:3, 18:3/18:4, 18:3/20:4, 18:3/20:5, 18:4/18:4, 18:4/20:4, 18:4/20:5, 20:4/20:5, and other novel molecular species as shown in Tables 2 and 3.

The novel lipid compositions, as disclosed herein, are highly-bioavailable sources of n-3 PUFAs and n-3 LC-PUFAs that enrich circulating, cellular and tissue levels in animals and humans. In addition, they have anti-inflammatory properties that block activities of numerous enzymes, such as cyclooxygenases, lipoxygenases, and cytochrome P450s that metabolize PUFAs and LC-PUFAs to pro-inflammatory mediators. Furthermore, the disclosed lipid compositions can inhibit the uptake of pro-inflammatory PUFAs and LC-PUFAs into cells and especially inflammatory cells.

ALA is an n-3 (or omega-3) essential fatty acid. ALA is found in many seeds and oils, including flaxseed, walnuts, chia, hemp, and many common vegetable oils. In terms of its structure, it is named all-cis-9,12,15-octadecatrienoic acid. In physiological literature, it is listed by its lipid number, 18:3, and (n-3). It is a carboxylic acid with an 18-carbon chain and three cis double bonds. The first double bond is located at the third carbon from the methyl end of the fatty acid chain, known as the n end. Thus, α-linolenic acid is an n-3 PUFA. It is an isomer of gamma-linolenic acid (GLA), an 18:3 (n-6) fatty acid (i.e., a polyunsaturated omega-6 fatty acid with three double bonds).

SDA (C18:4, an omega-3 fatty acid) is a target for the nutraceutical, pharmaceutical, aquaculture and livestock feed, and companion pet industries. SDA is more stable than DHA and EPA (longer shelf life, higher quality pure product). Importantly, because it bypasses the Δ6 desaturase step in n-3 LC-PUFA biosynthesis, SDA is much more efficiently converted to n-3 LC-PUFAs (compared to its precursor, alpha-linolenic acid, ALA). Human and animal studies reveal that SDA has a variety of health benefits, including reducing inflammation, hyperlipidemia, obesity and suppressing the growth of breast cancer (Whelan, J Nutr., January 2009, 139(1):5-10).

Omega-3 ETA (20:4, double bond positions 8, 11, 14, 17) is a very rare 20-carbon fatty acid that has been shown to be a potent inhibitor of inflammatory mechanisms induced by its omega-6 counterpart, 5,8,11,14 eicosatetraenoic acid (or omega-6 arachidonic acid). For example, it has been shown to inhibit enzymes involved in the uptake of omega-6 arachidonic acid into cells and the metabolism of omega-6 arachidonic acid to prostaglandins and thromboxanes via cyclooxygenase. See Simpoulos, Am. J Clin Nutr. 1991, 55:438-463; Ringbom et al., J Nat Prod. 2001 June; 64(6):745-9; and Neufeld et al., J. Lipid Res., 1984, 25:288-293. Consequently, it is a target for a therapeutic agent. For example, it has been suggested for use in the treatment of asthma. See U.S. Pat. No. 4,584,320, herein incorporated by reference in its entirety. However, because of its extreme rarity in nature, it has not moved forward as a pharmaceutical, nutraceutical, or feed additive.

EPA, eicosapentaenoic acid (20:5, double bond positions 5, 8, 11, 14, 17), is a primary long chain omega-3 fatty acid well established as a robust anti-inflammatory molecule. For example, a recent multi-year study of Vascepa (ethyl eicosapentaenoic acid), a prescription drug containing only EPA, was shown to reduce heart attack, stroke, and cardiovascular death by 25% relative to a placebo in those with statin-resistant hypertriglyceridemia. See Bhatt, New England Journal of Medicine. 380: 11-22; “Vascepa® (icosapent ethyl) 26% Reduction in Key Secondary Composite Endpoint of Cardiovascular Death, Heart Attacks and Stroke Demonstrated in REDUCE-IT™” (https://www.acc.org/latest-in-cardiology/clinical-trials/2018/11/08/22/48/reduce-it). Consequently, it is an important target as a therapeutic agent in addition to its inclusion in most omega-3 supplemented foods.

The chemical structures of MGDG with palmitic acid (16:0) in the sn-2 position and omega-3 fatty acid in the sn-1 position, ALA, SDA, n-3 ETA, and EPA, are depicted as follows:

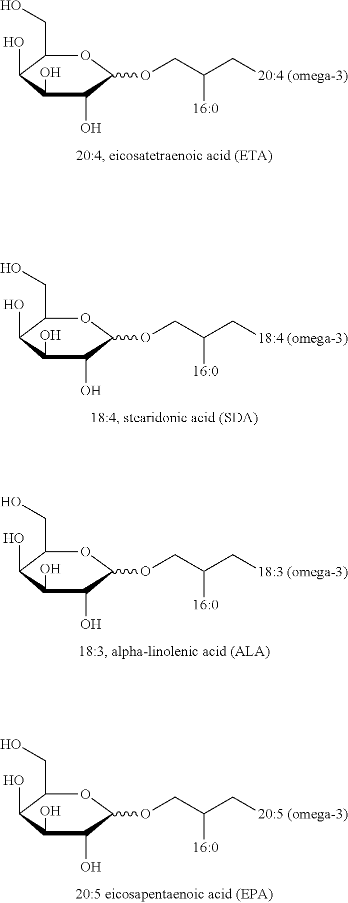

In some embodiments, a lipid composition of the present invention has a lipid profile characterized by a significant amount of n-3 PUFAs conjugated to polar lipids, such as phospholipids and/or glycolipids. In some embodiments, the glycolipids are present in a greater amount than other lipids such as phospholipids and triglycerides.

The fatty acyl chains of MGDG and DGDG can contain PUFAs (including n-3 FAs) in cyanobacteria. MGDG and DGDG are more soluble and can be extracted by supercritical fluid extraction. Importantly, recent human studies show n-3 PUFAs in galactolipids are more bioavailable in humans than phospholipids found in Krill oils. See Kagan et al., Lipids in Health and Disease, 12: 102 (2013). Sulfoquinovosyldiacylglycerol (SQDG), together with phosphatidylglycerol (PG), are major classes of the thylakoid membrane lipids in cyanobacteria and plant chloroplasts.

In some embodiments, the glycolipid comprises MGDG, DGDG, SQDG, or a combination thereof. In some embodiments, the omega-3 fatty acid is conjugated to (or complexed with) at least one of the sn-1 and sn-2 positions of MGDG, DGDG, or SGQG. In some embodiments, the omega-3 fatty acid is conjugated to both the sn-1 and sn-2 positions of MGDG, DGDG, or SGQG.

Chemical structures of example conjugates of ALA, SDA, omega-3 ETA, and/or EPA to MGDG are depicted as follows (DGDG species would have a second galactosyl group attached at the left):

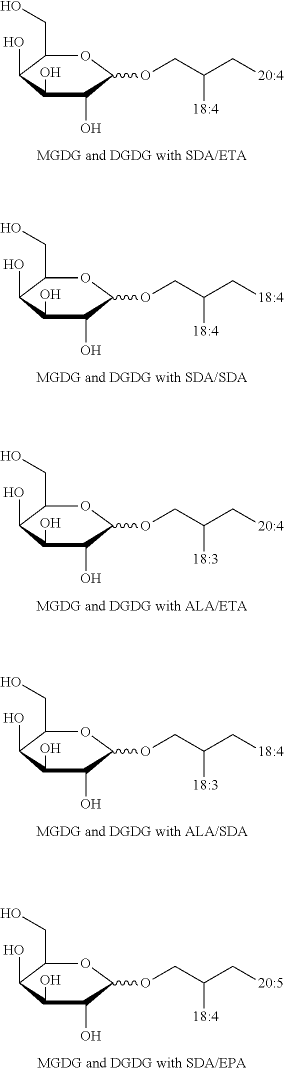

In some embodiments, the lipid composition of any one of the preceding claims, wherein at least 50% of SDA, omega-3 ETA, EPA or ALA is in the fraction of the omega-3 fatty acids conjugated to the MGDG, the DGDG, or both. In some embodiments, at least 60% of the SDA is in the fraction of the omega-3 fatty acid conjugated to the MGDG, the DGDG, or both. In some embodiments, at least 60% of the omega-3 ETA is in the fraction of the omega-3 fatty acid conjugated to the MGDG, the DGDG, or both. In some embodiments, the lipid composition comprises at least about 10% SDA, at least about 1% omega-3 ETA, at least about 0.1% EPA, or at least about 20% ALA, by weight of total fatty acid content. In some embodiments, a total amount of the SDA, the omega-3 ETA, EPA, and the ALA is at least about 20% by weight of total fatty acid content. In some embodiments, the lipid composition comprises between about 5% and about 40% ALA; between about 10% and about 30% SDA; and between about 1% and about 10% omega-3 ETA.

In some embodiments, a lipid composition having a particular lipid profile is provided. In some embodiments, a “profile” refers to a % of a given PUFA relative to the total fatty acid concentration or total omega-3 fatty acid concentration. In some embodiments, the lipid composition comprises at least about 9% (e.g., at least about 9, 10, 12, 14, 16, 18, 20, 21, 22, 23, 24, or 25%) ALA. In some embodiments, the lipid composition comprises at least about 8% (e.g., at least about 8, 10, 12, 14, 16, 18, 20, 21, 22, 23, or 24%) SDA. In some embodiments, the lipid composition comprises at least about 0.2% (e.g., at least about 0.2, 0.4, 0.8, 1.0, 1.2, 1.4, 1.6, 1.8, or 2, 2.5, 3, 3.5, 4, 4.5, or 5%) n-3 ETA.

In some embodiments, the lipid composition comprises at least about 23%, at least about 24%, at least about 25%, at least about 30%, or at least about 35% of the omega-3 fatty acids. In some embodiments, the lipid composition comprises at least about 9% ALA, at least about 15% (e.g., 16%, 17%, 18%, 19%, 20%, 21%, 22%, 23%, or 24%) SDA, and/or a detectable amount of omega-3 ETA, such as at least about 1, 2, 3, 4, or 5% omega-3 ETA. In some embodiments, the lipid composition comprises at least about 25% or more ALA (e.g., at least about 33% or more). In some embodiments, the lipid composition comprises a detectable amount of omega-3 ETA, such as at least about 1, 2, 3, 4, 5, 6, 7, 8, 9 or 10% omega-3 ETA. In some embodiments, the lipid composition comprises 1-10% omega-3 ETA.

In some embodiments, a total amount of the SDA, the omega-3 ETA, and the ALA is at least about 35% by weight of total fatty acid content. In some embodiments, a molar ratio of the omega-3 fatty acid to omega-6 fatty acid is between about 30:1 and about 90:1 (e.g., 35:1, 40:1, 45:1, 50:1, 55:1, 60:1, 65 1, 70:1, 75:1, 80:1, 85:1).

(2) Compositions

In another aspect, this disclosure also provides a composition, such as a pharmaceutical composition, comprising the lipid composition described above and a pharmaceutically acceptable carrier.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the invention, and are physiologically acceptable to the subject. Supplementary active compounds may also be incorporated into the compositions.

In some embodiments, the composition or the pharmaceutical composition may further include a therapeutic agent, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In some embodiments, the lipid composition is prepared in an administrable form selected from the group consisting of a pharmaceutical formulation, a nutritional formulation, a feed formulation, a dietary supplement, a medical food, a functional food, a beverage product, and combinations thereof.

Also within the scope of this disclosure is a food or drink additive comprising the lipid composition as described above. In some embodiments, the food or drink additive is formulated for food or drink selected from the group consisting of water, fruit juice, and vegetable juice, or for natural (or synthetic) flavoring and fragrance to prepare one or more foods. Also provided is a feed for use in aquaculture comprising the lipid composition described above.

The composition can be administered to any subject or patient in need thereof. Although preferred subjects are human, animals, especially domestic animals such as dogs, cats, horses, cattle, sheep, goats, and fowl, may also be treated with the composition. The amount of the active ingredients to be administered is chosen based on the amount which provides the desired dose to the patient in need of such treatment to alleviate symptoms or treat a condition (e.g., inflammation).

In some embodiments, the composition can be used in a cosmetic. Cosmetics include, but are not limited to, emulsions, creams, lotions, masks, soaps, shampoos, washes, facial creams, conditioners, make-ups, bath agents, and dispersion liquids. Cosmetic agents can be medicinal or non-medicinal.

In some embodiments, the composition can be an industrial composition. In some embodiments, the composition is a starting material for one or more industrial products. An industrial product includes, but is not limited to, a polymer, a photographic photosensitive material, a detergent, an industrial oil, or an industrial detergent.

Also within the scope of this disclosure is a product containing the lipid composition. In some embodiments, the product can be in packs in a form ready for administration, e.g., a blister pack, a bottle, syringes, foil packs, pouches, or other suitable containers. In some embodiments, the compositions are in concentrated form in packs, optionally with a diluent.

(3) Other Ingredients

In some embodiments, the lipid composition further comprises an additional agent such as emulsifier, emulsifying aid, stabilizer, antioxidant, ion antagonism, defoaming agent, natural (or synthetic) flavoring, natural (or synthetic) fragrance, and agents for balancing osmolarity.

a. Antioxidants

The lipid composition disclosed herein may further include an antioxidant. Antioxidants can be used to prevent or at least inhibit or mitigate the degradation of cannabinoids from oxidation. The antioxidant may be one or more selected from sodium sulfite, sodium hydrogen sulfite, sodium pyrosulfate, vitamin C esters thereof, and tocopherols and esters thereof, preferably vitamin C and mixed tocopherol; the emulsification aid may be selected from alkali metal salts with long-chain C16 to C20 fatty acids, preferably sodium salt thereof.

In some embodiments, antioxidants can be any one of: ethanol, polyethylene glycol 300, polyethylene glycol 400, propylene glycol, propylene carbonate, N-methyl-2-pyrrolidones, dimethylacetamide, dimethyl sulfoxide, hydroxypropyl-P-cyclodextrins, sulfobutylether-β-cyclodextrin, a-cyclodextrin, HSPC phospholipid, DSPG phospholipid, DMPC phospholipid, DMPG phospholipid, ascorbyl palmitate, butylated hydroxyanisole, butylated hydroxyanisole, propyl gallate, a-tocopherol, γ-tocopherol, propyl gallate, lecithin, Vitamin E tocopherol, sesamin, sesamol, sesamolin, alpha-tocopherol, ascorbic acid, ascorbyl palmitate, fumaric acid, malic acid, sodium metabisulfite, and EDTA. Specific antioxidant examples include, but are not limited to: Ascorbic Acid: 0.001 to 5% w/w of emulsion system, Vitamin E Tocopherol: 0.001 to 5% w/w of emulsion system, Tocopherol: 0.001 to 5% w/w of emulsion system, and combinations of ascorbic acid, vitamin E tocopherol, and tocopherol can be used for this invention.

b. Preservatives

The lipid composition disclosed herein may further include a preservative. Oil-in-water emulsions are aqueous in nature and susceptible to microbial growth. Preservatives can be used to prevent microbial spoilage. These preservatives include: methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzoate, sodium metabisulfite, propylene glycol, benzaldehyde, butylated hydroxytoluene, butylated hydroxyanisole, formaldehyde donors, essential oils, citric acid, monoglyceride, phenol, mercury components and any combination thereof.

Amongst useful preservatives include chelating agents some of which are listed above and other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3-PDTA); 2,2-ethylenedioxybis[ethyliminodi(acetic acid)] (EGTA); 1,10-bis(2-pyridylmethyl)-1,4,7,10-tetradecane (BPTETA); ethylenediamine (EDAMINE); Trans-1,2-diaminocyclohexane-N,N,N′,N′-tetraacetic acid (CDTA); ethylenediamine-N,N′-diacetate (EDDA); phenazine methosulphate (PMS); 2,6-Dichloro-indophenol (DCPIP); Bis(carboxymethyl)diaza-18-crown-6 (CROWN); porphine; chlorophyll; dimercaprol (2,3-Dimercapto-1-propanol); citric acid; tartaric acid; fumaric acid; malic acid; and salts thereof. The preservatives listed above are exemplary, but each preservative must be evaluated in each formulation, to assure the compatibility and efficacy of the preservative. Methods for evaluating the efficacy of preservatives in pharmaceutical formulations are known to those skilled in the art.

Additionally, the pH of the emulsion can be lowered to prevent or retard microbial growth. Lowering the pH below 4.0 is sufficiently low enough to prevent microbial growth for a minimum of 1 month.

c. Sweetener

In the instance where auxiliary sweeteners are utilized, the formulation may include those sweeteners well known in the art, including both natural and artificial sweeteners. Thus, additional sweeteners may be chosen from the following non-limiting list: water-soluble sweetening agents such as monosaccharides, disaccharides, and polysaccharides such as xylose, ribose, glucose, mannose, galactose, fructose, high fructose corn syrup, dextrose, sucrose, sugar, maltose, partially hydrolyzed starch, or corn syrup solids and sugar alcohols such as sorbitol, xylitol, mannitol, and mixtures thereof.

In general, the amount of sweetener will vary with the desired amount of sweeteners selected for a particular formulation. This amount will normally be 0.00%1 to about 90% by weight, per volume of the final composition, when using an easily extractable sweetener. The water-soluble sweeteners described above are preferably used in amounts of about 5% to about 70% by weight per volume, and most preferably from about 10% to about 50% by weight per volume of the final liquid composition. In contrast, the artificial sweeteners (e.g., sucralose, acesulfame K, and dipeptide based sweeteners) are used in amounts of about 0.005% to about 5.0% and most preferably about 0.01% to about 2.5% by weight per volume of the final liquid composition. These amounts are ordinarily necessary to achieve a desired level of sweetness independent from the flavor level achieved from flavor oils.

d. Flavoring Agents

Suitable flavorings include both natural and artificial flavors, and mints such as peppermint, menthol, artificial vanilla, cinnamon, various fruit flavors, both individual and mixed, essential oils (i.e., thymol, eucalyptol, menthol, and methyl salicylate) and the like are contemplated. The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. The flavorings are generally utilized in amounts that will vary depending upon the individual flavor, and may, for example, range in amounts of about 0.01 to about 3% by weight per volume of the final composition weight.

e. Colorants

The colorants useful in the present invention, include the pigments such as titanium dioxide that may be incorporated in amounts of up to about 1% by weight per volume, e.g., up to about 0.6% by weight per volume. Also, the colorants may include dyes suitable for food, drug, and cosmetic applications, and known as D&C and F.D. & C. dyes and the like. The materials acceptable for the foregoing spectrum of use are preferably water-soluble. Illustrative examples include indigoid dye, known as F.D. & C. Blue No. 2, which is the disodium salt of 5,5′indigotindisulfonic acid. Similarly, the dye known as F.D. & C. Green No. 1 comprises a triphenylmethane dye and is the monosodium salt of 4-[4-N-ethyl p-sulfobenzylamino)diphenylmethylene]-[1-(N-ethyl-N-p-sulfoniumbenzyl)-2,5-cyclohexadienimine]. A full recitation of all F.D. & C. and D. & C. and their corresponding chemical structures may be found in the Kirk-Othmer Encyclopedia of Chemical Technology, in Volume 5, at Pages 857-884, which text is accordingly incorporated herein by reference.

(4) Kits

A composition described herein can be provided in a kit. In one embodiment, the kit includes (a) a container that contains the composition, and optionally (b) informational material. The informational material can be descriptive, instructional, marketing or other material that relates to the methods described herein and/or the use of the agents for therapeutic benefit. In an embodiment, the kit also includes an additional therapeutic agent, as described above. For example, the kit includes a first container that contains the composition and a second container for the additional therapeutic agent.

The informational material of the kits is not limited in its form. In one embodiment, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

The kit can include one or more containers for the composition. In some embodiments, the kit contains separate containers, dividers or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading.

### B. Methods of Preparing the Lipid Compositions

(1) Recombinant Vectors

Provided in accordance with some embodiments of the presently disclosed subject matter is a recombinant vector comprising at least one nucleic acid sequence encoding a desaturase. In some embodiments, the recombinant vector further comprises a nucleic acid sequence encoding thylakoid-promoting protein Vipp1. In some embodiments, the recombinant vector comprises a nucleic acid sequence encoding Vipp1 or a variant (e.g., functional variant) thereof, a nucleic acid sequence encoding Δ6 desaturase or a variant (e.g., functional variant) thereof, a nucleic acid sequence encoding Δ15 desaturase (also referred to as omega-3 desaturase) or a variant (e.g., functional variant) thereof, or any combination thereof.

In some embodiments, the recombinant vector comprises a heterologous promoter operably linked to a nucleic acid sequence encoding Δ6 desaturase, a nucleic acid sequence encoding Δ15 desaturase, and/or a nucleic acid sequence encoding a thylakoid-promoting protein Vipp1. In some embodiments, the recombinant vector comprises a backbone sequence affording compatibility with a plurality of microorganisms.

In some embodiments, the recombinant vector comprises a nucleic acid sequence encoding Vipp1, a nucleic acid sequence encoding Δ6 desaturase, and a nucleic acid sequence encoding Δ15 desaturase operably oriented so that each polypeptide will be expressed. In some embodiments, each polypeptide is expressed in a suitable culture, where it produces a greater amount of one or more lipid compositions than does a control culture identical in all respects except that the polypeptides are not expressed or not expressed to a degree that they are expressed in the test culture.

In some embodiments, the recombinant vector comprises one or more nucleic acid sequence(s) comprising one or more sequences affording expression or transcription control, such as a promoter sequence, a repressor sequence, a terminator sequence, a transcription blocking sequence, and combinations thereof. In some embodiments, the recombinant vector comprises a nucleic acid sequence coding for a selectable marker, such as antibiotic resistance.

In some embodiments, the recombinant vector comprises a plasmid. In some embodiments, a recombinant vector is optimized for transformation of and/or expression in a microorganism. In some embodiments, the microorganism is a cyanobacterium, a diverse phylum of oxygenic phototrophs in the kingdom bacteria. In some embodiments, the cyanobacterium is in the order Gloeobacterales, Chroococcales, Nostocales, Oscillatoriales, Pleurocapsales, Prochlorales, or Stigonematales. In some embodiments, the cyanobacterium is unicellular. In some embodiments, the cyanobacterium is filamentous heterocystous. In some embodiments, the cyanobacterium is filamentous non-heterocystous. In some embodiments, the cyanobacterium is a freshwater strain. In some embodiments, the cyanobacterium is a marine strain. In some embodiments, the cyanobacterium is a species of Anabaena, Leptolyngbya, Lyngbya, Nostoc, Phormidium, Spirulina, Synechococcus, or Synechocystis. In some embodiments, the modified cyanobacterium is Anabaena sp. PCC7120, Synechococcus sp. PCC7002, or Leptolyngbya sp. strain BL0902.

In some embodiments, the nucleic acid sequence encoding a thylakoid-promoting protein Vipp1, the nucleic acid sequence encoding a Δ6 desaturase, and/or the nucleic acid sequence encoding a Δ15 desaturase is a natural gene sequence. In some embodiments, the nucleic acid sequence encoding a thylakoid-promoting protein Vipp1, the nucleic acid sequence encoding a Δ6 desaturase, and/or the nucleic acid sequence encoding a Δ15 desaturase is a synthetic gene sequence (e.g., a codon-optimized sequence).

In some embodiments, the Vipp1, Δ6 desaturase, and/or Δ15 desaturase is/are homologous with respect to a microorganism to be transformed with the recombinant vector. In some embodiments, the Vipp1, Δ6 desaturase, and/or Δ15 desaturase is/are heterologous with respect to a microorganism to be transformed with the recombinant vector.

In some embodiments, Vipp1 is encoded by a nucleic acid sequence comprising SEQ ID NO: 1. However, the nucleic acid sequence can comprise any other sequence that encodes an amino acid sequence comprising SEQ ID NO:5. In some embodiments, the desaturase is encoded by a nucleic acid sequence comprising a sequence selected from the group comprising SEQ ID NOs: 2 and 3 or another nucleic acid sequence that encodes an amino acid sequence as set forth in SEQ ID NO:6 or SEQ ID NO:7. In some embodiments, Vipp1, Δ6 desaturase, and Δ15 desaturase are each encoded by a single nucleic acid sequence, such as a nucleic acid sequence comprising SEQ ID NO: 4. However, the nucleic acid sequence can be any other single nucleic sequence that encodes each of SEQ ID NOs: 5, 6, and 7, wherein the nucleic acid sequences encoding SEQ INOs: 5, 6, and 7 can be arranged in any order within the larger nucleic acid sequence.

Synthetic vipp1

The 40 base-pair sequence upstream of the vipp1 gene is identical to the sequence upstream of the PCC 6803 atpE gene. atpE encodes the F₁Fₒ ATP synthase subunit c, which is present in a higher copy number than the other subunits, while being translated from a polycistronic operon. The sequence upstream of the atpE gene was shown to be responsible for the enhanced translation needed to provide the higher copy number of subunit c. The corresponding region upstream of atpE from E. coli has previously been used to increase expression of genes from plasmids in E. coli.

(SEQ ID NO: 1)
ctcgagtaaactttactgtcgttttgttagctaaaaggaaaaaattATGG
GCTTCCTGGACCGTCTGGGCCGTGTCGTGAAAGCGAACCTGAATGATATG
GTGTCGAAAGCAGAAGACCCGGAAAAAATCCTGGAACAGGCAGTCGCTGA
TATGGGCGAAAGCCTGGTCCAACTGCGTCAGTCTGTGGCGCGTGCGATTG
CGGCCCAGAAGAAAACCGAACAGCAACTGATCAAAAACCAAACCGAAGCG
ACCACGTGGCAGAAGAAAGCGGAACTGGCCATTAAAAATGGTCGTGAAGA
TCTGGCACGCGAAGCTCTGGTTCGTAAGAAAACCTTTGCAGACACGGCAG
CTGTCCTGCAGCAACAGCTGACGCAACAGAACGCCCAAGTTAAAACCCTG
AAAGAAAATCTGCTGGCACTGGAAAGTAAAATCCAGGAAGCTAAAACCAA
GAAAGATATGCTGAAAGCACGCGCTAACGCGGCCAAAGCGAATGCCCAAC
TGCAGAGTACGATGAACAATATTGATACCAGCTCTGCGATGTCCGCCTTT
GAACGTATGGAAGACAAAATCATGGAACTGGAAGCACAGTCTGAAGCTAC
CAACGTGCTGGGCAGTGCGTCCCTGGATCAAGAATTCGCGCAGCTGGAAG
CCTCAAATTCGATTGATGACGAACTGGCCATGCTGAAAGCAGCTTCACAG
GAAGCACCGGCTCTGGAAGCGGCCAAAATGGTTGATGAAATGGAAGAAGT
GTCGGACACCCCGGTTGAAACGCCGGAACCGACCGACGCGGACTTTGCGG
CTGTTGATGCGGAACTGGAAGCACTGCGTACCCAGATGAAAAACCTGTAA
aagcttggcgcgcc
Synthetic Acyl-lipid Δ6-desaturase gene
(SEQ ID NO: 2)
catATGCTGACGGCAGAACGTATCAAGTTTACCCAGAAGCGTGGCTTTCG
TCGTGTCCTGAACCAACGTGTGGATGCGTATTTTGCTGAACATGGTCTGA
CCCAGCGTGATAACCCGTCAATGTATCTGAAAACGCTGATTATCGTCCTG
TGGCTGTTCTCGGCGTGGGCCTTTGTGCTGTTCGCACCGGTTATTTTTCC
GGTCCGCCTGCTGGGTTGCATGGTTCTGGCAATCGCTCTGGCGGCCTTTT
CATTCAATGTCGGCCATGATGCAAACCACAATGCTTATAGCTCTAACCCG
CATATTAATCGTGTTCTGGGCATGACCTACGACTTCGTCGGTCTGAGTTC
CTTTCTGTGGCGTTATCGCCACAACTACCTGCATCACACCTATACGAATA
TTCTGGGCCATGATGTGGAAATCCACGGCGACGGTGCCGTTCGTATGAGC
CCGGAACAGGAACATGTGGGTATCTACCGCTTTCAGCAATTCTATATTTG
GGGCCTGTACCTGTTTATCCCGTTCTATTGGTTTCTGTATGATGTCTACC
TGGTGCTGAACAAAGGTAAGTACCATGACCACAAAATTCCGCCGTTTCAA
CCGCTGGAACTGGCATCTCTGCTGGGCATCAAGCTGCTGTGGCTGGGCTA
TGTGTTCGGTCTGCCGCTGGCGCTGGGCTTTTCAATTCCGGAAGTCCTGA
TCGGTGCCTCGGTGACCTATATGACGTACGGCATTGTGGTTTGTACCATC
TTCATGCTGGCTCATGTTCTGGAAAGCACCGAATTTCTGACGCCGGATGG
CGAATCTGGTGCAATTGATGACGAATGGGCTATTTGCCAGATCCGTACCA
CGGCGAACTTCGCCACCAACAATCCGTTCTGGAACTGGTTTTGTGGCGGT
CTGAATCACCAAGTGACGCATCACCTGTTTCCGAACATTTGCCATATCCA
CTATCCGCAGCTGGAAAACATCATCAAGGACGTTTGTCAAGAATTCGGTG
TGGAATATAAAGTTTACCCGACCTTTAAGGCAGCTATTGCGAGTAATTAC
CGCTGGCTGGAAGCGATGGGCAAGGCCTCCTAA
Synthetic Acyl-lipid Δ15 desaturase gene
(SEQ ID NO: 3)
ATGCAATCCACCGTTCGTTCTCCGGGTAGCCGTGAAAGCCTGCGCCAAGA
CCTGCCGTTTACGCTGAAAGACGTGAAAGCCGCCATTCCGGATTATTGCT
TCCAGCCGAGCGTGTTTCGTTCTCTGGCGTACTTTTTCCTGGATATCGGT
ATTATCGCAGGCCTGTATGCTATTGCGGCCTACCTGGACTCTTGGTTTTT
CTATCCGATCTTTTGGTTCGCACAGGGTACGATGTTTTGGGCTCTGTTCG
TGGTTGGCCATGATTGTGGCCACGGTAGCTTCTCTCGCAGTAAATTTCTG
AACGACCTGATCGGTCATCTGAGCCACACCCCGATTCTGGTTCCGTTTCA
TGGCTGGCGTATTTCACACCGCACCCATCACTCGAACACGGGTAATATCG
ATACCGACGAAAGCTGGTATCCGATTCCGGAATCTAAATACGATCAGATG
GGCTTCGCGGAAAAACTGGTCCGTTTTTATGCGCCGCTGATTGCCTATCC
GATCTACCTGTTTAAACGCAGTCCGGGTCGTGGTCCGGGTTCCCATTTCT
CACCGAAATCGCCGCTGTTTAAACCGGCGGAACGCAATGATATTATCCTG
TCCACGGCAGCTATTATCGCCATGGTCGGTTTTCTGGGCTGGTTCACCGT
GCAATTTGGTCTGCTGGCCTTCGTTAAATTTTATTTCGTCCCGTACGTGA
TTTTTGTTATCTGGCTGGATCTGGTTACGTATCTGCATCACACCGAAGCA
GACATCCCGTGGTACCGTGGTGATGACTGGTATTACCTGAAAGGCGCTCT
GAGTACCATTGATCGCGACTATGGTATTTTCAACGAAATCCATCACAATA
TTGGCACGCATGTCGCCCATCACATCTTTCACACCATTCCGCATTACCAC
CTGAAAGATGCGACCGAAGCCATCAAACCGCTGCTGGGCGACTATTACCG
TGTGAGCCATGCACCGATTTGGCGTAGCTTTTTCCGCTCCCAGAAAGCGT
GTCACTACATCGCCGACCAAGGCAGCCACCTGTATTATCAACCGAAAAAA
TAA

For expression of all three genes:
Δ6 desaturase+vipp1+Δ15 desaturase construction

The restriction enzyme sites used for the construction are in bolded double underlined and in italics.

Sub-sequence including the coding sequence for Δ6
desaturase (i.e. 4-1084 of SEQ ID NO: 2)
(SEQ ID NO: 4)
ATGCTGACGGCAGAACGTATCAAGTTTACCCAGAAGCGTGGCTTTCGTCG
TGTCCTGAACCAACGTGTGGATGCGTATTTTGCTGAACATGGTCTGACCC
AGCGTGATAACCCGTCAATGTATCTGAAAACGCTGATTATCGTCCTGTGG
CTGTTCTCGGCGTGGGCCTTTGTGCTGTTCGCACCGGTTATTTTTCCGGT
CCGCCTGCTGGGTTGCATGGTTCTGGCAATCGCTCTGGCGGCCTTTTCAT
TCAATGTCGGCCATGATGCAAACCACAATGCTTATAGCTCTAACCCGCAT
ATTAATCGTGTTCTGGGCATGACCTACGACTTCGTCGGTCTGAGTTCCTT
TCTGTGGCGTTATCGCCACAACTACCTGCATCACACCTATACGAATATTC
TGGGCCATGATGTGGAAATCCACGGCGACGGTGCCGTTCGTATGAGCCCG
GAACAGGAACATGTGGGTATCTACCGCTTTCAGCAATTCTATATTTGGGG
CCTGTACCTGTTTATCCCGTTCTATTGGTTTCTGTATGATGTCTACCTGG
TGCTGAACAAAGGTAAGTACCATGACCACAAAATTCCGCCGTTTCAACCG
CTGGAACTGGCATCTCTGCTGGGCATCAAGCTGCTGTGGCTGGGCTATGT
GTTCGGTCTGCCGCTGGCGCTGGGCTTTTCAATTCCGGAAGTCCTGATCG
GTGCCTCGGTGACCTATATGACGTACGGCATTGTGGTTTGTACCATCTTC
ATGCTGGCTCATGTTCTGGAAAGCACCGAATTTCTGACGCCGGATGGCGA
ATCTGGTGCAATTGATGACGAATGGGCTATTTGCCAGATCCGTACCACGG
CGAACTTCGCCACCAACAATCCGTTCTGGAACTGGTTTTGTGGCGGTCTG
AATCACCAAGTGACGCATCACCTGTTTCCGAACATTTGCCATATCCACTA
TCCGCAGCTGGAAAACATCATCAAGGACGTTTGTCAAGAATTCGGTGTGG
AATATAAAGTTTACCCGACCTTTAAGGCAGCTATTGCGAGTAATTACCGC
TGGCTGGAAGCGATGGGCAAGGCCTCCTAAagctcgagtaaactttactg
tcgttttgttagctaaaagga
Sub-sequence including the coding sequence for
Vipp1 (i.e. 47-849 of SEQ ID NO: 1),
aaaaattATGGGCTTCCTGGACCGTCTGGGCCGTGTCGTGAAAGCGAACC
TGAATGATATGGTGTCGAAAGCAGAAGACCCGGAAAAAATCCTGGAACAG
GCAGTCGCTGATATGGGCGAAAGCCTGGTCCAACTGCGTCAGTCTGTGGC
GCGTGCGATTGCGGCCCAGAAGAAAACCGAACAGCAACTGATCAAAAACC
AAACCGAAGCGACCACGTGGCAGAAGAAAGCGGAACTGGCCATTAAAAAT
GGTCGTGAAGATCTGGCACGCGAAGCTCTGGTTCGTAAGAAAACCTTTGC
AGACACGGCAGCTGTCCTGCAGCAACAGCTGACGCAACAGAACGCCCAAG
TTAAAACCCTGAAAGAAAATCTGCTGGCACTGGAAAGTAAAATCCAGGAA
GCTAAAACCAAGAAAGATATGCTGAAAGCACGCGCTAACGCGGCCAAAGC
GAATGCCCAACTGCAGAGTACGATGAACAATATTGATACCAGCTCTGCGA
TGTCCGCCTTTGAACGTATGGAAGACAAAATCATGGAACTGGAAGCACAG
TCTGAAGCTACCAACGTGCTGGGCAGTGCGTCCCTGGATCAAGAATTCGC
GCAGCTGGAAGCCTCAAATTCGATTGATGACGAACTGGCCATGCTGAAAG
CAGCTTCACAGGAAGCACCGGCTCTGGAAGCGGCCAAAATGGTTGATGAA
ATGGAAGAAGTGTCGGACACCCCGGTTGAAACGCCGGAACCGACCGACGC
GGACTTTGCGGCTGTTGATGCGGAACTGGAAGCACTGCGTACCCAGATGA
AAAACCTGTAAaagcttaaaatataa
Sub-sequence including the coding sequence for
Δ15 desaturase (i.e. 1-1053 of SEQ ID NO: 3),
gtaggagataaaaacATGCAATCCACCGTTCGTTCTCCGGGTAGCCGTGA
AAGCCTGCGCCAAGACCTGCCGTTTACGCTGAAAGACGTGAAAGCCGCCA
TTCCGGATTATTGCTTCCAGCCGAGCGTGTTTCGTTCTCTGGCGTACTTT
TTCCTGGATATCGGTATTATCGCAGGCCTGTATGCTATTGCGGCCTACCT
GGACTCTTGGTTTTTCTATCCGATCTTTTGGTTCGCACAGGGTACGATGT
TTTGGGCTCTGTTCGTGGTTGGCCATGATTGTGGCCACGGTAGCTTCTCT
CGCAGTAAATTTCTGAACGACCTGATCGGTCATCTGAGCCACACCCCGAT
TCTGGTTCCGTTTCATGGCTGGCGTATTTCACACCGCACCCATCACTCGA
ACACGGGTAATATCGATACCGACGAAAGCTGGTATCCGATTCCGGAATCT
AAATACGATCAGATGGGCTTCGCGGAAAAACTGGTCCGTTTTTATGCGCC
GCTGATTGCCTATCCGATCTACCTGTTTAAACGCAGTCCGGGTCGTGGTC
CGGGTTCCCATTTCTCACCGAAATCGCCGCTGTTTAAACCGGCGGAACGC
AATGATATTATCCTGTCCACGGCAGCTATTATCGCCATGGTCGGTTTTCT
GGGCTGGTTCACCGTGCAATTTGGTCTGCTGGCCTTCGTTAAATTTTATT
TCGTCCCGTACGTGATTTTTGTTATCTGGCTGGATCTGGTTACGTATCTG
CATCACACCGAAGCAGACATCCCGTGGTACCGTGGTGATGACTGGTATTA
CCTGAAAGGCGCTCTGAGTACCATTGATCGCGACTATGGTATTTTCAACG
AAATCCATCACAATATTGGCACGCATGTCGCCCATCACATCTTTCACACC
ATTCCGCATTACCACCTGAAAGATGCGACCGAAGCCATCAAACCGCTGCT
GGGCGACTATTACCGTGTGAGCCATGCACCGATTTGGCGTAGCTTTTTCC
GCTCCCAGAAAGCGTGTCACTACATCGCCGACCAAGGCAGCCACCTGTAT
TATCAACCGAAAAAATAAcgggcgcgcc

SEQ ID NO:4 is an exemplary construction of a nucleic acid sequence that encodes each of the Δ6 desaturase, the Δ15 desaturase, and Vipp1. Other constructions can include the coding sequences for the two desaturases and vipp1 in other orders and/or with other non-coding intervening sequences. Other constructions can also include more than one copy of the coding sequence(s) of any or all of the Δ6 desaturase, the Δ15 desaturase, and Vipp1.

Amino Acid Sequences:

Amino Acid Sequence for Synthetic Vipp1
(SEQ ID NO: 5)
MGFLDRLGRVVKANLNDMVSKAEDPEKILEQAVADMGESLVQLRQSVARA
IAAQKKTEQQLIKNQTEATTWQKKAELAIKNGREDLAREALVRKKTFADT
AAVLQQQLTQQNAQVKTLKENLLALESKIQEAKTKKDMLKARANAAKANA
QLQSTMNNIDTSSAMSAFERMEDKIMELEAQSEATNVLGSASLDQEFAQL
EASNSIDDELAMLKAASQEAPALEAAKMVDEMEEVSDTPVETPEPTDADF
AAVDAELEALRTQMKNL
Amino Acid Sequence for Synthetic Acyl-lipid Δ6
desaturase
(SEQ ID NO: 6)
MLTAERIKFTQKRGFRRVLNQRVDAYFAEHGLTQRDNPSMYLKTLIIVLW
LFSAWAFVLFAPVIFPVRLLGCMVLAIALAAFSFNVGHDANHNAYSSNPH
INRVLGMTYDFVGLSSFLWRYRHNYLHHTYTNILGHDVEIHGDGAVRMSP
EQEHVGIYRFQQFYIWGLYLFIPFYWFLYDVYLVLNKGKYHDHKIPPFQP
LELASLLGIKLLWLGYVFGLPLALGFSIPEVLIGASVTYMTYGIVVCTIF
MLAHVLESTEFLTPDGESGAIDDEWAICQIRTTANFATNNPFWNWFCGGL
NHQVTHHLFPNICHIHYPQLENIIKDVCQEFGVEYKVYPTFKAAIASNYR
WLEAMGKAS
Amino Acid Sequence for Synthetic Acyl-Lipid Δ15
desaturase
(SEQ ID NO: 7)
MQSTVRSPGSRESLRQDLPFTLKDVKAAIPDYCFQPSVFRSLAYFFLDIG
IIAGLYAIAAYLDSWFFYPIFWFAQGTMFWALFVVGHDCGHGSFSRSKFL
NDLIGHLSHTPILVPFHGWRISHRTHHSNTGNIDTDESWYPIPESKYDQM
GFAEKLVRFYAPLIAYPIYLFKRSPGRGPGSHFSPKSPLFKPAERNDIIL
STAAIIAMVGFLGWFTVQFGLLAFVKFYFVPYVIFVIWLDLVTYLHHTEA
DIPWYRGDDWYYLKGALSTIDRDYGIFNEIHHNIGTHVAHHIFHTIPHYH
LKDATEAIKPLLGDYYRVSHAPIWRSFFRSQKACHYIADQGSHLYYQPKK

In some embodiments, a recombinant vector in accordance with the presently disclosed subject matter comprises a coding sequence comprising a nucleotide sequence of any of SEQ ID NOs: 1-4; or a coding sequence comprising a nucleotide sequence substantially identical to any of SEQ ID NOs: 1-4. In some embodiments, a recombinant vector in accordance with the presently disclosed subject matter comprises a coding sequence comprising a nucleotide sequence that is 70%, 75%, 80%, 85%, 90%, 91%, 92%, 93%, 94%, 95%, 96%, 97%, 98% or 99% identical to any of SEQ ID NOs: 1-4. In some embodiments, a recombinant vector in accordance with the presently disclosed subject matter comprises a coding sequence comprising a nucleotide sequence that encodes an amino acid sequence that is 70%, 75%, 80%, 85%, 90%, 91%, 92%, 93%, 94%, 95%, 96%, 97%, 98% or 99% identical to any of SEQ ID NOs: 5-7.

The representative recombinant vector is a 13,611 bp plasmid that includes the pAM44148 expression vector describe in Taton et al. (2012) that contains both the lac repressor and trc promoter from E. coli, as well as synthetic genes for the Δ6 and Δ15 desaturases and Vipp1.

| Feature | Function |
|---|---|
| Δ6 desaturase | Synthetic gene (amino acid sequence from |
| | Synechocystis sp. PCC 6803) |
| Δ15 desaturase | Synthetic gene (amino acid sequence from |
| | Synechococcus sp. PCC 7002). |
| | Also known as Δ15 desaturase. |
| Vipp1 | Vesicle-inducing protein in plastids. Important for |
| | synthesis of thylakoid membranes. Synthetic gene, |
| | amino acid sequence from Synechococcus sp. |
| | PCC 7002 |
| aadA | Aminoglycoside-3: adenyltransferase. Confers |
| | resistance to spectinomycin and streptomycin |
| | antibiotics. |
| lacI | A repressor protein introduced to allow control of |
| | protein expression from the trc promoter |
| Ori | Sequence used for replication of the plasmid DNA |
| mob A, B, C and | Genes encoding proteins required for replication |
| repA, B, C | of the plasmid in cells |
| lacO | DNA sequence to which LacI binds and blocks |
| | transcription |
| AttB1, 2 | DNA sequences used or cloning genes into the |
| | pAM4418 expression plasmid |
| trpA and rrnB | DNA sequences that block continued transcription, |
| T2 terminators | helping to limit unwanted expression from |
| | the plasmid |
| SP6, T7 | DNA sequences used for PCR and sequencing |

(2) Modified Microorganisms

Cyanobacteria are proven producers of biomaterials, including the n-6 18C-PUFA γ-linolenic acid (GLA, 18:3), generally requiring only sun, water, and trace nutrients (nitrate) to produce large quantities of biomass (19, 20). Additionally, cyanobacteria may provide n-3 PUFAs and LC-PUFAs largely in a polar glycolipid form that is thought to be more bioavailable than fish and seed-based sources (21). Due to differences in digestive routes and physical forms (polar vs. nonpolar lipids) of n-3 PUFA- and LC-PUFA-containing complex lipids, the bioavailability of diverse forms vary considerably. To date, most n-3 PUFAs or LC-PUFAs have been provided to humans and animals complexed to non-polar triglycerides from seed oils or marine fats. However, there are numerous problems with highly-enriched triglyceride formulations, including the fact that large quantities of such concentrates are typically needed to achieve effective circulating and tissue (especially brain) levels of PUFAs and LC-PUFAs (22). To overcome these obstacles, ethyl esters, free fatty acids, re-esterified triglycerides or phospholipids (in the case Krill oil) have been formulated, although with varying degrees of success. Cyanobacteria (and dark green plants with abundant chloroplasts) have thylakoid membranes that contain large quantities of the galactose-containing glycolipids MGDG and DGDG (23). Importantly, there is a selective pancreatic lipase (PLRP2) that mobilizes fatty acids from MGDG and DGDG (24), and initial rodent and human studies suggest that ingestion of LC-PUFAs with or complexed to these glycolipids improves their bioavailability (21, 25).

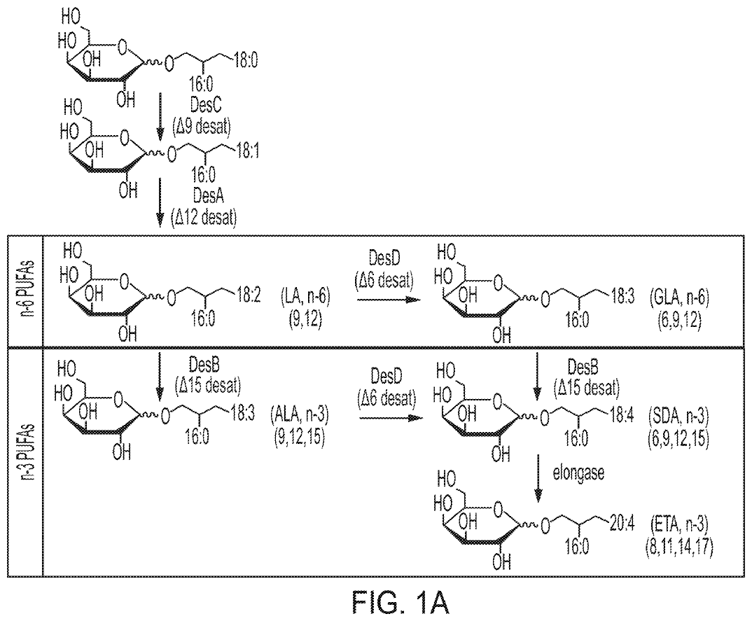

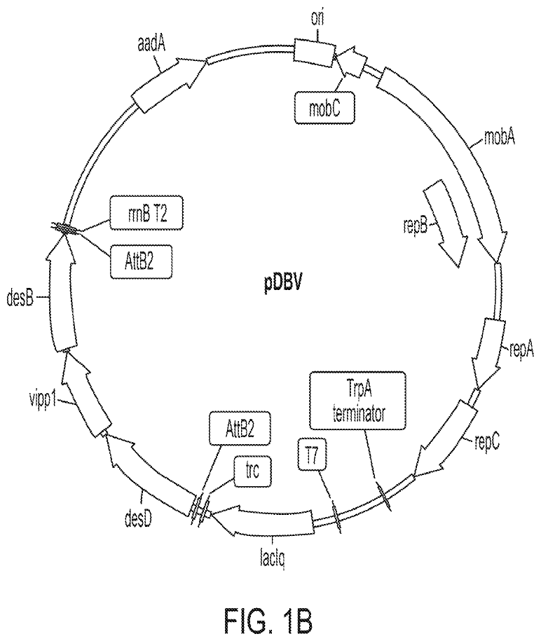

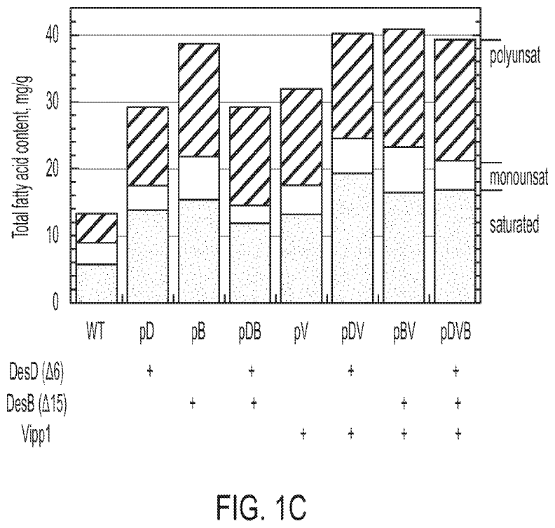

As the prokaryotic precursors of chloroplasts, cyanobacteria are biologically simpler than plants and algae, and genetic manipulation is generally more feasible, enabling metabolic reprogramming by engineering. Importantly for the purpose of this work, they also contain acyl-lipid desaturases, and “Group 4” cyanobacteria have the critical four desaturases (DesC, DesA, DesB, and DesD, FIG. 1A) necessary to convert stearic acid (18:0) to SDA (26). However, as opposed to acyl-CoA or acyl-ACP desaturases, these desaturases act directly on fatty acids within the glycolipids, and these lipids account for ˜80% of total lipids in thylakoid membranes (27, 28).

With these understandings, this disclosure provides genetically modified cyanobacteria strains that augment the expression of three genes, desB and desD encoding acyl-lipid desaturases (known as Δ15 and Δ6 desaturases, respectively), and/or vipp1 encoding a thylakoid membrane enhancing protein. This disclosure demonstrated that it is possible to markedly increase the capacity of cyanobacteria to produce SDA and omega-3 ETA complexed to highly bioavailable MGDG and DGDG molecular species.

In some embodiments, the presently disclosed subject matter provides a modified microorganism, for example, an engineered cyanobacterium, as a source of omega-3 PUFAs and omega-6 PUFAs, such as but not limited to ALA, SDA, and omega-3 ETA. In some embodiments, the modified microorganism comprises the recombinant vector, as described above. In some embodiments, the modified microorganism comprises a first exogenous gene encoding Vipp1, wherein the modified microorganism further comprises at least a second exogenous gene encoding a desaturase; wherein the modified microorganism produces a lipid in a greater amount than does a control microorganism identical in all respects except that it does not include the first exogenous gene encoding Vipp1 and the second exogenous gene encoding a desaturase. In some embodiments, the modified microorganism comprises at least two exogenous genes encoding a desaturase, wherein each gene encodes a different desaturase. In some embodiments, the desaturase is a Δ6 desaturase or a Δ15 desaturase. In some embodiments, the first desaturase is a Δ6 desaturase and the second desaturase is a Δ15 desaturase. In some embodiments, the various gene constructs, as disclosed herein, are integrated into the host genome.

In some embodiments, the nucleic acid sequence encoding Vipp1 or a variant (e.g., functional variant) thereof, the nucleic acid sequence encoding a Δ6 desaturase or a variant (e.g., functional variant) thereof, and/or the nucleic acid sequence encoding a Δ15 desaturase or a variant (e.g., functional variant) thereof is a natural gene sequence. In some embodiments, the nucleic acid sequence encoding Vipp1 or a variant (e.g., functional variant) thereof, the nucleic acid sequence encoding a Δ6 desaturase or a variant (e.g., functional variant) thereof, and/or the nucleic acid sequence encoding a Δ15 desaturase or a variant (e.g., functional variant) thereof is a synthetic gene sequence (e.g., codon-optimized sequence).

In some embodiments, the Vipp1, Δ6 desaturase, and/or Δ15 desaturase is/are homologous with respect to the modified microorganism. In some embodiments, Vipp1, Δ6 desaturase, and/or Δ15 desaturase is/are heterologous with respect to the modified microorganism.

In another aspect, this disclosure further provides a method of producing the lipid composition, as described above. The method comprises (a) culturing a modified microorganism comprising at least one exogenous gene encoding a desaturase in a culture medium under conditions in which the at least one exogenous gene encoding a desaturase is expressed; and (b) enriching the cultured modified microorganism from the culture medium, wherein the cultured modified microorganism produces a greater amount of the lipid than does a culture comprising a control microorganism identical in all respects except that it does not include the at least one exogenous gene encoding the desaturase.

In some embodiments, the at least one exogenous gene comprises a first gene encoding a Δ6 desaturase and a second gene encoding a Δ15 desaturase. In some embodiments, the desaturase comprises a polypeptide sequence having at least about 75% identity to SEQ ID NO: 6 or 7 or the polypeptide sequence of SEQ ID NO: 6 or 7. In some embodiments, the desaturase is encoded by a nucleic acid sequence having at least about 75% identity to a nucleic acid sequence of SEQ ID NOs: 2-3 or comprising a nucleic acid sequence of SEQ ID NOs: 2-3.

In some embodiments, the modified microorganism further comprises an exogenous gene encoding thylakoid-promoting protein Vipp1. In some embodiments, the Vipp1 comprises a polypeptide sequence having at least about 75% identity to SEQ ID NO: 5 or comprising the polypeptide sequence of SEQ ID NO: 5. In some embodiments, the Vipp1 is encoded by a nucleic acid sequence having at least about 75% identity to SEQ ID NO: 1 or comprising SEQ ID NO: 1.

In some embodiments, this disclosure also provides a method of culturing a lipid-producing microorganism. In some embodiments, the method comprises: providing a culture of a modified microorganism that comprises an exogenous gene encoding Vipp1 and at least one exogenous gene encoding a desaturase in a suitable culture medium under conditions in which the exogenous gene encoding the Vipp1 and the exogenous gene encoding the desaturase are expressed. In some embodiments, the culture produces a greater amount and a greater proportion of selected n-3 PUFAs such as ALA, SDA, and omega-3 ETA than does a culture comprising a control microorganism identical in all respects except that it does not include the exogenous gene encoding the Vipp1 and at least one exogenous gene encoding a desaturase. In some embodiments, the modified microorganism comprises the recombinant vector, as described above.

In some embodiments, the method further comprises extracting/isolating the lipids and the omega-3 fatty acid from biomass of the cultured modified microorganism.

In some embodiments, Vipp1 is encoded by a nucleic acid sequence comprising SEQ ID NO: 1, or by a coding sequence comprising a nucleotide sequence that is 70%, 75%, 80%, 85%, 90%, 91%, 92%, 93%, 94%, 95%, 96%, 97%, 98% or 99% identical to SEQ ID NO: 1 and/or any nucleic acid sequence that encodes SEQ ID NO:5 or an amino acid sequence at least about 70%, 75%, 80%, 85%, 90%, 91%, 92%, 93%, 94%, 95%, 96%, 97%, 98% or 99% identical to SEQ ID NO:5. In some embodiments, the desaturase is encoded by a nucleic acid sequence comprising a sequence selected from the group comprising SEQ ID NOs: 2 and 3, or by a coding sequence comprising a nucleotide sequence that is 70%, 75%, 80%, 85%, 90%, 91%, 92%, 93%, 94%, 95%, 96%, 97%, 98% or 99% identical to SEQ ID NOs: 2 or 3 and/or to a nucleic acid sequence that encodes SEQ ID NO:6 or SEQ ID NO:7 or an amino acid sequence at least about 70%, 75%, 80%, 85%, 90%, 91%, 92%, 93%, 94%, 95%, 96%, 97%, 98% or 99% identical to SEQ ID NO:6 or SEQ ID NO:7. In some embodiments, the Vipp1, Δ6 desaturase, and Δ15 desaturase are each encoded by a nucleic acid sequence comprising SEQ ID NO: 4 or by a coding sequence comprising a nucleotide sequence that is 70%, 75%, 80%, 85%, 90%, 91%, 92%, 93%, 94%, 95%, 96%, 97%, 98% or 99% identical to SEQ ID NO: 4. In some embodiments, the Vipp1, Δ6 desaturase and Δ15 desaturase are each encoded by another nucleic acid that encodes each of SEQ ID NO:5, SEQ ID NO: 6, and SEQ ID NO: 7, wherein the coding sequences for SEQ ID NOs: 5, 6, and 7 are arranged in any order within the larger sequence.

In some embodiments, the lipid composition is produced by a modified microorganism. In some embodiments, the microorganism is a cyanobacterium, a diverse phylum of oxygenic phototrophs in the kingdom bacteria. In some embodiments, the cyanobacterium is in the order Gloeobacterales. In some embodiments, the cyanobacterium is in the order Chroococcales. In some embodiments, the cyanobacterium is in the order Nostocales. In some embodiments, the cyanobacterium is in the order Oscillatoriales. In some embodiments, the cyanobacterium is in the order Pleurocapsales. In some embodiments, the cyanobacterium is in the order Prochlorales. In some embodiments, the cyanobacterium is in the order Stigonematales.

In some embodiments, the cyanobacterium is unicellular. In some embodiments, the cyanobacterium is filamentous heterocystous. In some embodiments, the cyanobacterium is filamentous non-heterocystous. In some embodiments, the cyanobacterium is a freshwater strain. In some embodiments, the cyanobacterium is a marine strain. In some embodiments, the cyanobacterium is a species of Anabaena, Leptolyngbya, Lyngbya, Nostoc (e.g., Nostoc commune), Phormidium (e.g., Phormidium valderianum), Spirulina, Synechococcus or Synechocystis.

In some embodiments, the modified cyanobacterium is Anabaena sp. PCC7120, Synechococcus sp. PCC7002, or Leptolyngbya sp. strain BL0902.

In some embodiments, the modified microorganisms are used as nutraceuticals (including but not limited to pharmaceuticals, dietary supplements, medical foods, and functional foods) and/or additives to food, including food for humans and for animal feed (e.g., feed for fish, such as Tilapia, and/or for other animals, such as fowl, swine, and cattle). Thus, in some embodiments, the modified microorganisms can be used in aquaculture. Representative formulation techniques and administration approaches are disclosed in U.S. Pat. No. 8,343,753, which is incorporated herein by reference in its entirety.

### C. Methods of Use of the Lipid Compositions

In another aspect, this disclosure further provides a method for the prophylactic and/or therapeutic treatment of a disease or condition, in particular a cardiovascular or inflammatory disease or a condition involving a psychological or neurodevelopmental disorder. The method comprises administering enterally or parentally a dose (e.g., therapeutically effective amount) of the lipid composition, the composition, or the pharmaceutical composition, as described above, to a subject in need thereof. In some embodiments, the subject is a mammal, such as human.

This disclosure also provides a method for treating a mammalian disease in a subject by administrating to the subject a therapeutically effective amount of the lipid composition, the composition, or the pharmaceutical composition, as described above, to the subject in need thereof. In some embodiments, the mammalian diseases that are treated include, but are not limited to, cardiovascular diseases and inflammatory diseases. In other embodiments, the cardiovascular diseases to be treated include, but are not limited to, hypertriglyceridemia, coronary heart disease, stroke, acute myocardial infarction, and atherosclerosis. In further embodiments, the inflammatory diseases to be treated include, but are not limited to, asthma, arthritis, allergic rhinitis, psoriasis, atopic dermatitis, inflammatory bowel diseases, Alzheimer's disease, Crohn's disease, and allergic rhinoconjunctivitis. In additional embodiments, the mammalian diseases to be treated include psychiatric disorders. Psychiatric disorders include, but are not limited to, depression, bipolar disorder, schizophrenia. In addition, the compositions of the presently disclosed subject matter can be used to maintain and/or enhance cognitive function and prevent and/or treat brain inflammation.

In yet another aspect, this disclosure additionally provides a method for treating a human having omega-3 fatty acid deficiency. The method comprises administering to the human an effective dosage amount of the lipid composition, the composition, or the pharmaceutical composition, as described above.

In some embodiments, the human has a condition selected from the group consisting of a systemic inflammatory response syndrome, a respiratory distress syndrome, a nutritional and/or dietary cause of liver disease, an iatrogenic cause of liver disease, a pathological cause of liver disease, an immune modulation, head trauma, postoperative surgical stress, a myocardial infarction, cystic fibrosis, and a combination thereof.

In some embodiments, the human is in need of rapidly supplementing omega-3 fatty acids to improve metabolic syndrome, or to benefit from the efficacy of omega-3 fatty acids in modulating inflammation, prevention of premature birth, myocardial ischemia or infarction, transient local cerebral ischemia or stroke, autoimmunity, and thrombotic diseases, organ transplantation, acute phase response, acute respiratory distress syndrome, inflammatory bowel syndrome, and hypertriglyceridemia.

The terms “subject,” “individual,” and “patient” are used interchangeably herein to refers to any animal (e.g., avian, fish or mammal), including, but not limited to, humans, non-human primates, birds, and the like, which is to be the recipient of a particular treatment. Illustrative avians according to the presently disclosed subject matter include chickens, ducks, turkeys, geese, quail, pheasant, ratites (e.g., ostrich), domesticated birds (e.g., parrots and canaries), and birds in ovo. Fish of the presently disclosed subject matter include, but are not limited to, salmon, tilapia, carp, trout, bream, catfish, bass, sturgeon, and the like. Mammals of the presently disclosed subject matter include, but are not limited to, canines, felines, bovines, caprines, equines, ovines, porcines, rodents (e.g., rats and mice), lagomorphs, primates (including non-human primates), humans, and the like, and mammals in utero. Any mammalian subject in need of being treated according to the presently disclosed subject matter is suitable. According to some embodiments of the presently disclosed subject matter, the mammal is a non-human mammal. In some embodiments, the mammal is a human subject. Mammalian subjects of both genders and at any stage of development (i.e., neonate, infant, juvenile, adolescent, adult) can be treated according to the presently disclosed subject matter.

As used herein, the phrase “therapeutically effective amount” refers to an amount of a compound or composition that is sufficient to produce the desired effect, which can be a therapeutic or agricultural effect. The therapeutically effective amount will vary with the application for which the compound or composition is being employed, the microorganism and/or the age and physical condition of the subject, the severity of the condition, the duration of the treatment, the nature of any concurrent treatment, the pharmaceutically or agriculturally acceptable carrier used, and like factors within the knowledge and expertise of those skilled in the art. An appropriate “therapeutically effective amount” in any individual case can be determined by one of ordinary skill in the art by reference to the pertinent texts and literature and/or by using routine experimentation. (See, for example, for pharmaceutical applications, Remington, The Science And Practice of Pharmacy (9th Ed. 1995).)

### D. Definitions

### Abbreviations

LC: Long chain;
desB and DesB, Δ15 acyl lipid desaturase (gene and protein, respectively);
desD and DesD: Δ6 acyl lipid desaturase (gene and protein, respectively);
Vipp1: Vesicle-inducing protein in plastids 1;
DesA: Δ12 acyl lipid desaturase;
DesC: Δ9 acyl lipid desaturase;
SDA: stearidonic acid (18:4 n-3, 18 carbons with 4 double bonds, 6Z,9Z,12Z,15Z-octadecatetraenoic acid);
ALA: alpha-linolenic acid (18:3 n-3, 9Z,12Z,15Z-octadecatrienoic acid);
ETA: eicosatetraenoic acid (20:4 n-3, 8Z,11Z,14Z,17Z-eicosatetraenoic acid, also known as ω-3-Arachidonic acid);
GLA: gamma-linolenic acid (18:3 n-6, 6Z,9Z,12Z-octadecatrienoic acid);
LA: linoleic acid (18:2 n-6, 9Z,12Z-octadecadienoic acid);
MGDG: monogalactosyldiacylglycerol;
DGDG: digalactosyldiacylglycerol;
SQDG: sulfoquinovosyldiacylglycerol;
pDBV: plasmid derived from pAM4418 containing genes encoding DesD, DesB and Vipp1;
CoA: coenzyme A;
ACP: acyl carrier protein;
BL0902: Leptolyngbya sp. strain BL0902;
7002: Synechococcus sp. PCC7002;
7120: Anabaena sp. PCC7120;
GC-FID: gas chromatography-flame ionization detection;
COX1 and COX2: cyclooxygenases 1 and 2;
FAME: fatty acid methyl esters.

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “lipid” includes phospholipids, free fatty acids, esters of fatty acids, triacylglycerols, diacylglycerols, monoacylglycerols, lysophospholipids, soaps, phosphatides, sterols and sterol esters, carotenoids, xanthophylls (e.g., oxycarotenoids), hydrocarbons, and other lipids known to one of ordinary skill in the art.

The term “neutral lipid” includes triacylglycerols, diacylglycerols, monoacylglycerols, free fatty acids, sterol esters, etc.

The term “polar lipid” includes phospholipids, such as phosphatidylinositol, phosphatidylserine, phosphatidylcholine, phosphatidylglycerol and phosphatidylethanolamine, polar glycolipids, galactolipids, and the like.

In connection with a lipid composition of the presently disclosed subject matter, a “profile” refers to the distribution of particular chemical species within the composition. In some embodiments, a “profile” refers to a % of a given PUFA relative to the total fatty acid concentration.

The term “non-human feed” or “non-human food” refers to any food intended for non-human animals, whether for fish; commercial fish; ornamental fish; fish larvae; bivalves; mollusks; crustaceans; shellfish; shrimp; larval shrimp; artemia; rotifers; brine shrimp; filter feeders; amphibians; reptiles; or mammals, such as dogs, cats, guinea pigs, rabbits, rats, mice, horses, monkeys, cows, cattle, pigs, sheep, and the like. An animal feed includes, but is not limited to, an aquaculture feed, a domestic animal feed including pet feed, a zoological animal feed, a work animal feed, a livestock feed, and combinations thereof.

The terms “polypeptide,” “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

“Exogenous gene” refers to a nucleic acid sequence that codes for the expression of an RNA and/or protein that has been introduced into a cell (e.g., by transformation/transfection), and is also referred to as a “transgene.” A cell comprising an exogenous gene can be referred to as a recombinant cell, into which additional exogenous gene(s) can be introduced. The exogenous gene can be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene can be present in more than one copy in the cell. An exogenous gene can be a natural gene, e.g., excised from a natural source, or can be synthesized.

As used herein, the term “variant” refers to a first molecule that is related to a second molecule (also termed a “parent” molecule). The variant molecule can be derived from, isolated from, based on or homologous to the parent molecule.

As applied to proteins, a variant polypeptide can have an entire amino acid sequence identity with the original parent polypeptide or can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence. Polypeptide variants include polypeptides comprising the entire parent polypeptide, and further comprising additional fused amino acid sequences. Polypeptide variants also include polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide, and yield functionally identical polypeptides, including additions of non-functional peptide sequence. In other aspects, the variant polypeptides of the invention change the biological activity of the parent molecule. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

As used herein, the term “promoter” or “regulatory sequence” refers to a nucleic acid sequence which is required for expression of a gene product operably linked to the promoter/regulatory sequence. In some instances, this sequence may be the core promoter sequence, and in other instances, this sequence may also include an enhancer sequence and other regulatory elements that are required for expression of the gene product. The promoter or regulatory sequence may, for example, be one that expresses the gene product in a tissue-specific manner. An “inducible” promoter is a nucleotide sequence that, when operably linked with a polynucleotide that encodes or specifies a gene product, causes the gene product to be produced in a cell substantially only when an inducer that corresponds to the promoter is present in the cell.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product(s).” If the polynucleotide is derived from genomic DNA, expression may include splicing of the mRNA in a eukaryotic cell.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

The term “percent sequence identity,” in the context of two or more amino acid or nucleic acid sequences, refers to two or more sequences or subsequences that are the same or have a specified percentage of amino acid residues or nucleotides that are the same, when compared and aligned for maximum correspondence, as measured using a sequence comparison algorithm or by visual inspection. For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted using the NCBI BLAST software (ncbi.nlm.nih.gov/BLAST/) set to default parameters. For example, to compare two nucleic acid sequences, one may use blastn with the “BLAST 2 Sequences” tool Version 2.0.12 (Apr. 21, 2000) set at the following default parameters: Matrix: BLOSUM62; Reward for match: 1; Penalty for mismatch: −2; Open Gap: 5 and Extension Gap: 2 penalties; Gap.times.drop-off: 50; Expect: 10; Word Size: 11; Filter: on. For a pairwise comparison of two amino acid sequences, one may use the “BLAST 2 Sequences” tool Version 2.0.12 (Apr. 21, 2000) with blastp set, for example, at the following default parameters: Matrix: BLOSUM62; Open Gap: 11 and Extension Gap: 1 penalties; Gap.times.drop-off 50; Expect: 10; Word Size: 3; Filter: on.

“Recombinant” is a cell, nucleic acid, protein or vector that has been modified due to the introduction of an exogenous nucleic acid or the alteration of a native nucleic acid. Thus, e.g., recombinant cells can express genes that are not found within the native (non-recombinant) form of the cell or express native genes differently than those genes are expressed by a non-recombinant cell (e.g., overexpress a gene). Recombinant cells can, without limitation, include recombinant nucleic acids that encode for a gene product or for suppression elements such as mutations, knockouts, antisense, interfering RNA (RNAi) or dsRNA that reduce the levels of active gene product in a cell. A “recombinant nucleic acid” is a nucleic acid originally formed in vitro, in general, by the manipulation of nucleic acid, e.g., using polymerases, ligases, exonucleases, and endonucleases, using chemical synthesis, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of the presently disclosed subject matter. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of the presently disclosed subject matter. Similarly, a “recombinant protein” is a protein made using recombinant techniques, i.e., through the expression of a recombinant nucleic acid.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

The terms “prevent,” “preventing,” “prevention,” “prophylactic treatment” and the like refer to reducing the probability of developing a disorder or condition in a subject (e.g., plant), who does not have, but is at risk of or susceptible to developing a disorder or condition.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### E. Examples

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

This example describes the materials and methods used in EXAMPLES 2-4 below.

### Molecular Biology Approaches

Recombinant plasmids encoding 1-3 cyanobacterial genes of interest were derived from the pAM4418 expression vector, a broad host range, E. coli-cyanobacteria shuttle plasmid that confers resistance to streptomycin and spectinomycin and contains both the lacI^{q} repressor and the trc promoter from E. coli. The plasmid contains a Gateway recombination cassette that allows for gene transfer from a Gateway donor plasmid. Coding sequences for the following cyanobacterial proteins were synthesized by GenScript (codon-optimized for expression in E. coli): (1) DesD, the acyl-lipid Δ6-desaturase from Synechocystis sp. PCC 6803, (2) DesB, the acyl-lipid methyl-end (Δ15 or omega-3) desaturase from Synechococcus sp. PCC 7002, and (3) Vesicle-inducing protein in plastids (Vipp1) from Synechococcus sp. PCC 7002. From these sequences, the following seven expression plasmids were generated:
pD: encodes DesD, Δ6 desaturase
pB: encodes DesB, Δ15 desaturase
pDB: encodes both DesD and DesB
pV: encodes Vipp1
pDV: encodes DesD and Vipp1
pBV: encodes DesB and Vipp1
pDBV: encodes DesD, DesB and Vipp1

In each case, coding sequences were first PCR-amplified and cloned into the Gateway donor plasmid, pENTR/SD/D-topo (Invitrogen), which provides an upstream Shine-Dalgarno sequence (ribosome-binding site) that is known to function in cyanobacteria. Sequences of all seven plasmid inserts were verified and transferred into pAM4418 using the Gateway recombination system (Invitrogen) and Invitrogen's LR Clonase II Enzyme Mix, with verification of positive clones by restriction digestion. Details of the construction of each plasmid vary and are as follows.

In order to create the engineered pENTR plasmids to generate pD and pB, desD and desB, respectively, were PCR amplified using primers which added the sequence CACC before the initiating ATG codon and a Xho1 restriction site following the termination codon, enabling directional cloning of the PCR product into pENTR/SD/D-topo. For the next two constructs, pDV and pBV, the downstream XhoI restriction sites after each desaturase-encoding gene, in combination with the AscI site in the pENTR/SD/D-topo plasmid, provided sites for insertion of vipp1; the vipp1-containing fragment was excised from the Genscript plasmid using XhoI and Asc1, then ligated following each of the two desaturase genes into the pENTR/SD/D-topo clones described above. To create the pENTR plasmid encoding only Vipp1 (to generate pV), vipp1 from Genscript was PCR amplified as described above for desD and desB to enable directional cloning of the PCR product into pENTR/SD/D-topo.

In order to create pDBV, the desB sequence from GenScript was amplified by PCR to introduce a HindIII site plus a ribosome-binding site on the 5′ end and an Asc1 restriction site on the 3′ end. The PCR product was digested with HindIII and AscI and ligated into the pENTR/SD/D-topo plasmid already containing desD and vipp1, digested with the same two restriction enzymes.

Finally, the pENTR plasmid used to generate pDB, encoding only DesD and DesB, was derived from the pENTR plasmid containing all three genes (above) by digestion with HindIII and XhoI to remove the Vipp1-encoding gene, filling in the ends with dNTPs and DNA polymerase (Klenow fragment), then ligating the blunt ends together.

### Genetic Engineering, Growth, and Harvest of Cyanobacterial Strains

The seven pAM4418-derived plasmids were used to transform E. coli DH10B cells containing the conjugal and helper plasmids, pRL443 and pRL623, respectively. Transformants were grown overnight in rich LB media, washed with fresh LB, and resuspended in BG-11 media as a 10-fold concentrated stock. Cultures of the three host cyanobacteria (Leptolyngbya sp. strain BL0902, Anabaena sp. PCC7120 in BG-11 media and Synechococcus sp. PCC7002 in Medium A (55)) were grown to late exponential phase, harvested by centrifugation and washed twice with fresh media, before resuspension as a 4-fold concentrated stock. Cyanobacterial suspensions were sonicated in a bath for 10 min to reduce the length of the multicellular strands, then mixed with DH10B transformants. Cell mixtures were centrifuged, resuspended in 200 μL of BG-11 media, incubated for 1 h at 30° C., then spread on BG-11/5% LB agar plates. After incubation for 24 h in low light at 30° C., cells were washed and spread on BG-11 agar containing 2 μg/mL spectinomycin and streptomycin. After 7-10 days incubation at 30° C. under illumination [˜20-30 μmol photons/(m²·s)], single colonies were restreaked onto a fresh antibiotic-containing plate and incubated for 5-7 days. Bacteria scraped from the plate were transferred to 30 mL of BG-11 in a 250 mL conical flask, grown for 5 days at 30° C. (with shaking at 120 rpm and illumination), then harvested by centrifugation (5,000 rpm for 10 min). For Synechococcus sp. PCC7002, Medium A replaced BG-11 media in all steps of the conjugation and growth. Cell pellets were stored frozen at −80° C., then dried by lyophilization and weighed before the analysis of lipid content.

### Characterization of the Fatty Acid Content of Cyanobacteria Lipids

Lyophilized cell pellets were extracted utilizing a modified Bligh/Dyer for total fatty acid analysis (˜2 mg/sample). For total fatty acid analysis, solvents were evaporated under a stream of nitrogen in the presence of a fatty acid internal standard (triheptodecanoin, 10 μg). The dried extract was then subjected to base hydrolysis and derivatization in the presence of boron trifluoride (5 min, 100° C.) to form fatty acid methyl esters (FAME) following a modification of the protocol by Metcalfe et al. (56) as previously described (57, 58). FAMEs were analyzed on an Agilent J&W DB-23 column (30 m×0.25 mm ID, film thickness 0.25 m) using HP 5890 gas chromatography (GC) with a flame ionization detector (FID). Individual fatty acids were identified by their elution times relative to authenticated fatty acid standards, and fatty acid quantities were determined by their abundance relative to the internal standard.

For lipidomics analysis, total lipid extracts derived from 2 mg lyophilized biomass from wild type and pDBV-modified strains of Leptolyngbya sp. strain BL0902 and Synechococcus sp. PCC7002 were dried as above and dissolved in 100 μL isopropyl alcohol/methanol (50:50) for LC-MS/MS analysis. Samples (10% of total per injection) were analyzed on a high resolution Q Exactive HF Hybrid Quadrupole—Orbitrap Mass Spectrometer equipped with a heated electrospray ionization (HESI)-II source (Thermo Scientific, Rockford, IL) and a Vanquish Horizon UHPLC system (Thermo Scientific, Rockford, IL), with source parameters as follows: sheath gas flow rate, 40 L/min; auxiliary gas flow rate, 5 L/min; spray voltage, 3.5 kV with negative mode; capillary temperature, 350° C.; S-lens RF voltage, 75 V.

Chromatographic separation was achieved on an Accucore C30 column (2.6 μm, 3 mm×150 mm, Thermo Scientific, Rockford, IL) with linear gradient elution consisting of mobile phases A (water/acetonitrile=40:60) and B (isopropyl alcohol/acetonitrile=90:10) at 0.35 mL/min. Both mobile phases contained 0.1% formic acid and 10 mM ammonium formate, and the gradient was from 40% B at 0 min to 95% B at 30 min.

MS spectra were acquired by data-dependent scans in positive and negative mode. A survey scan was performed at MS1 level to identify top ten most abundant precursor ions followed by MS2 scans where productions were generated from selected ions. High-energy collisional dissociation (HCD) was utilized for ion fragmentation with stepped collision energy of 25/30 eV and 30/50/100 eV in each positive and negative polarity (79). The dynamic exclusion option was enabled during data-dependent scans to enhance compound identification in complex mixtures. Acquired spectra were processed using LipidSearch software v4.1 (Thermo Scientific, Rockford, IL) with the selection of following classes of lipids: lysophosphatidylcholine (LPC), phosphatidylcholine (PC), lysophosphatidylethanolamine (LPE), phosphatidylethanolamine (PE), lysophosphatidylserine (LPS), phosphatidylserine (PS), lysophosphatidylglycerol (LPG), phosphatidylglycerol (PG), lysophosphatidylinositol (LPI), phosphatidylinositol (PI), lysophosphatidic acid (LPA), phosphatidic acid (PA), sphingomyelin (SM), phytosphingosine (phSM), monoglyceride (MG), diglyceride (DG), triglyceride (TG), fatty acid (FA), (O-acyl)-1-hydroxy fatty acid (OAHFA), cardiolipin (CL), sphingoshine (So), sphingoshine phosphate (SoP), glucosylsphingoshine (SoGI), monoglycosylceramide (CerG1), diglycosylceramide (CerG2), triglycosylceramide (CerG3), ceramides (Cer), monosialotetrahexosylganglioside (GM2), cholesteryl ester (ChE), zymosteryl (ZyE), stigmasteryl ester (StE), sitosteryl ester (SiE), coenzymes (Co), monogalactosylmonoacylglycerol (MGMG), monogalactosyldiacylglycerol (MGDG), digalactosylmonoacylglycerol (DGMG), digalactosyldiacylglycerol (DGDG), sulfoquinovosylmonoacylglycerol (SQMG), and sulfoquinovosyldiacylglycerol (SQDG). Parameters for the product search workflow were: precursor mass tolerance, 5 ppm; product mass tolerance, 5 ppm; production intensity threshold, 1.0% relative to precursor; matching score threshold, 2.0. All peak areas were normalized to the total ion current (the total area under the curve in the chromatogram).

### Example 2

Generation of Plasmids and Engineered Cyanobacteria, and Analyses of Total Fatty Acid Content.

To augment the production of SDA-containing glycolipids and potentially n-3 LC-PUFA products of additional elongation and desaturation reactions such as ETA, EPA, and DHA, two genes encoding fatty acid desaturases, desB and desD, were targeted for enhanced expression. These desaturases act directly on 18C PUFAs within glycolipids and insert a new double bond, either (i) 3 carbons from the methyl end (DesB, also known as o3 desaturase or Δ15 desaturase), or (ii) 6 carbons from the carboxylate end (DesD, also known as Δ6 desaturase). FIG. 1A illustrates the pathway by which linoleic acid (LA; 18:2 n-6) bound to glycolipids is converted by the Δ15 desaturase (DesB) to ALA (18:3 n-3). ALA can then be acted upon by Δ6 desaturase (DesD) to insert a fourth double bond and form SDA (18:4 n-3). Alternatively, LA in glycolipids can be converted to GLA (18:3 n-6) by Δ6 desaturase activity and then GLA to SDA by Δ15 desaturase.

Previous studies reveal that these desaturase reactions occur within thylakoid membranes, and a thylakoid membrane formation enhancer gene, vipp1 (which encodes Vesicle-inducing protein in plastids or Vipp1, also known as IM30) (27, 28), was included to potentially boost levels of newly-synthesized PUFAs formed by the enhanced desaturase system. All three synthetic genes (desB, desD, and vipp1) encoding the authentic cyanobacterial protein products were incorporated into the expression plasmid pAM4418 (FIG. 1B) first described by Taton and colleagues (19), either singly or in combination with one or two other genes. The constructs were conjugated into Leptolyngbya sp. strain BL0902 (hereafter designated BL0902), a freshwater, filamentous cyanobacterium noted for its excellent growth characteristics and high lipid and especially LA content (19). No obvious deleterious effects on growth were observed in any of the seven exconjugants.

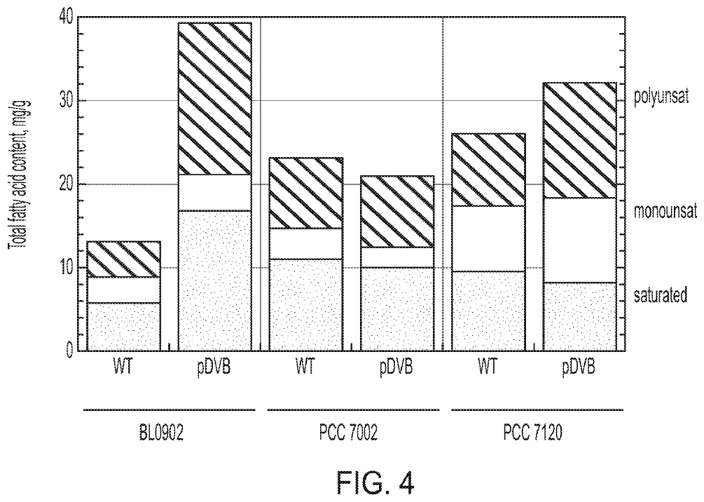

All plasmid-bearing cells of BL0902 showed a marked elevation in both saturated and polyunsaturated fatty acids (with monounsaturated levels varying) compared with the wild type (FIG. 1C); the total fatty acid content of the seven exconjugants ranged from 2 to 2.5-fold greater after conjugation. The three gene-containing plasmid pDBV (FIG. 1B) increased the total fatty acid content from about 13 to 39 mg/g dry weight and total PUFAs from 5.8 to 16.9 mg/g dry weight (FIG. 1C and Table 1A). Addition of vipp1 to all desaturase-expressing exconjugants elevated the total fatty acid content (FIG. 1C). Additional experiments were carried out to determine the impact of pDBV addition on two other cyanobacterial strains, Synechococcus sp. PCC7002 and Anabaena sp. PCC7120 (subsequently referred to as 7002 and 7120). Strain 7002 engineered with pDBV showed no increase in total lipids, while 7120 exhibited a modest increase of ˜23% (FIG. 4).

TABLE 1
Summary of pBV and pDBV plasmid
effects on cyanobacterial fatty acids.
Part A. Total Fatty Acid Contents (mg/g dry wt)^{a}

| Host strain | Fatty acid parameter | Wild type (WT) | WT + pBV | WT + pDBV |
|---|---|---|---|---|
| Leptolyngbya sp. | Total fatty acids (FAs) | 13.2 ± 7.6 | 40.9 ± 3.0 | 39.4 ± 1.8 |
| BL0902 | Saturated FAs | 4.3 ± 2.9 | 17.6 ± 1.7 | 18.2 ± 0.3 |
| | Monounsaturated FAs | 3.1 ± 1.9 | 6.8 ± 0.5 | 4.3 ± 0.4 |
| | Polyunsaturated FAs | 5.8 ± 2.9 | 16.4 ± 0.8 | 16.9 ± 1.4 |

Part B. C18 and C20 Omega-3 Fatty Acid Contents (mol % of total fatty acids)

| Host strain | Fatty acid parameter | WT | WT + pBV | WT + pDBV |
|---|---|---|---|---|
| Leptolyngbya sp. | Total SDA | 0 | 0 | 26.6 ± 1.0% |
| BL0902 | Total SDA + ALA + ETA | 23.8 ± 1.6% | 39.0 ± 1.8% | 40.1 ± 3.2% |
| | Ratio omega-3/omega-6 | 1.2 | 57 | 69 |
| Synechococcus | Total SDA | 0 | n.d.^{b} | 17.3 ± 2.1% |
| sp. PCC7002 | Total SDA + ALA + ETA | 7.1 ± 2.0% | n.d. | 40.3 ± 1.6% |
| | Ratio omega-3/omega-6 | 0.2 | n.d. | 16 |

^{a}Measured by gas chromatography and flame ionization of methyl ester-derivatized fatty acyl groups (FAME analysis), reported as mean ± standard deviation; n = 3 for all samples except n = 5 for wild type Leptolyngbya.
^{b}n.d. = not determined.

### Example 3

Modulation of Individual Fatty Acids in Engineered Strains.

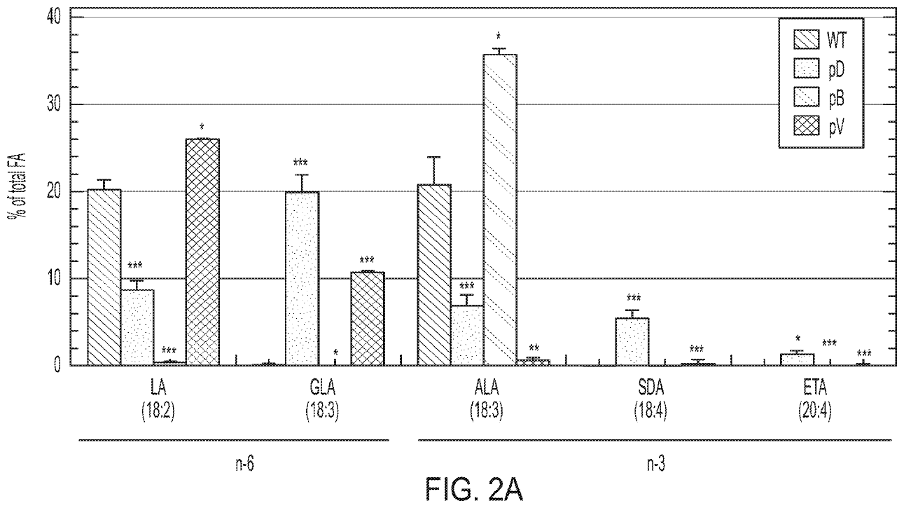

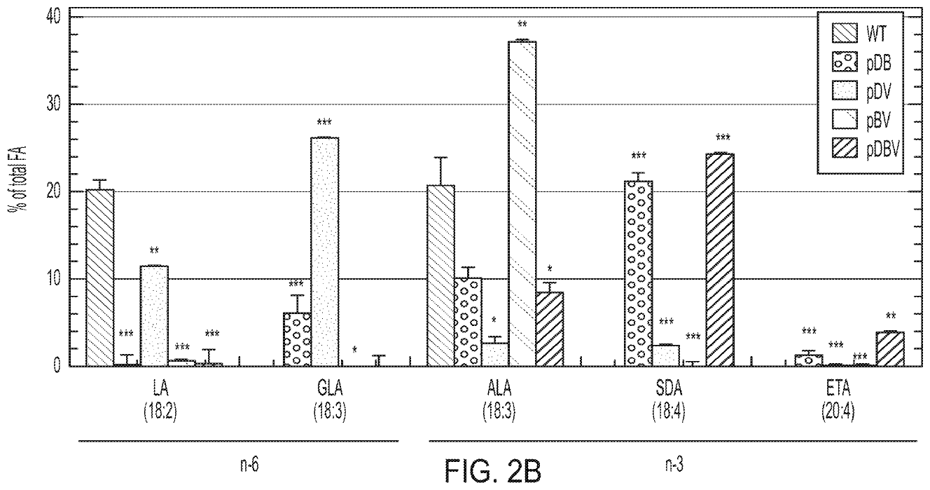

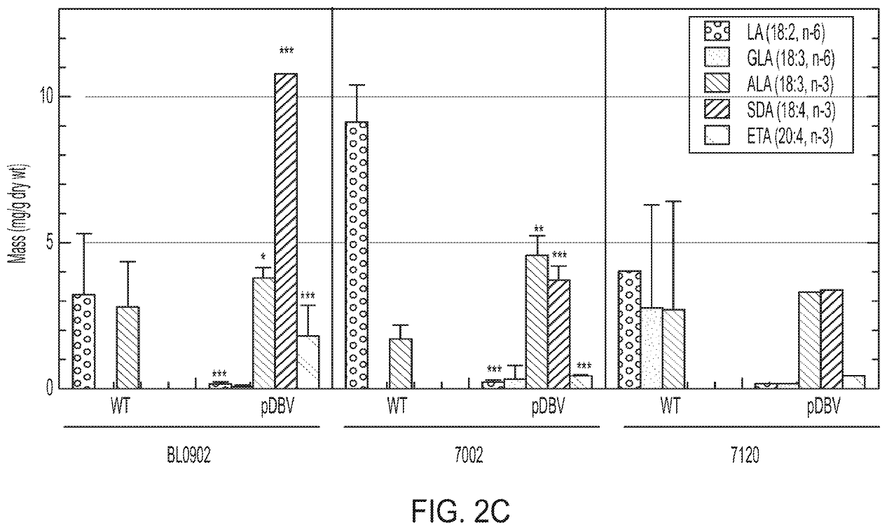

Individual fatty acids (expressed as mg/g dry weight or percentage of total fatty acids) from the total lipid extracts of wild type and exconjugants of the three cyanobacterial strains were analyzed by gas chromatography-flame ionization detection (GC-FID) and mass levels compared. FIG. 2A illustrates the effect of inclusion of just one of the three genes on n-6 and n-3 PUFAs in BL0902. When only desD (encoding the Δ6 desaturase) is present on the plasmid, the organism now produces some amount of all five 18C and 20C PUFAs, with GLA predominating (consistent with the conversion of LA to GLA catalyzed by DesD). Maximal SDA is seen when both desaturases are included on the plasmid (FIG. 2B), particularly when vipp1 is also included (SDA as a percent of total FAs is not much changed between pDB and pDBV, but the total mass increases significantly with the latter). With no desD included, expression of desB provided by the plasmid has a strong effect and leads to considerable accumulation of ALA at the expense of LA in BL0902 constructs pB and pBV (gold in FIG. 2A and dark blue in FIG. 2B, Table 1) consistent with the Δ15 desaturase activity of DesB (FIG. 1A). Importantly, the full three-gene plasmid shifted the profile to the greatest quantities of SDA and ETA, with some ALA remaining but no LA or GLA (red in FIGS. 2B and 2C). Total SDA contents reached 26.6±1.0 mol % of total fatty acids in the pDBV exconjugant, and the total n-3 PUFAs, including LC-PUFAs (ALA, SDA, and ETA) reached 40% of the lipid content in BL0902 (Table 1). The remarkable shift from n-6 to n-3 PUFA-containing lipids in the pDBV exconjugant was limited from producing additional SDA and ETA primarily by the unavailability of precursor substrates, LA and GLA. With such a depletion of LA and GLA, the pDBV exconjugant produced a ratio of n-3 to n-6 PUFAs of 69:1 (Table 1).

The two other strains of cyanobacteria (7002 and 7120) were tested with and without pDBV, and like BL0902, wild type strains contained no detectable SDA or ETA, but both were produced upon addition of pDBV (FIG. 2C). Quantities of SDA produced by 7002 and 7120 were 31-35% of that generated by the engineered BL0902 strain (FIG. 2C). Although not depicted in FIG. 2, small amounts of the n-3 LC-PUFAs EPA (20:5, n-3) and docosapentaenoic acid (DPA; 22:5, n-3) were also observed in some of the engineered (but not wild type) strains. Mass quantities of 0.03±0.02 and 0.19±0.05 mg/g dry weight were obtained for EPA in engineered 7002 and BL0902, respectively, while DPA was observed at 0.25 mg/g dry weight in only 7120.

### Example 4

Lipidomics Analysis

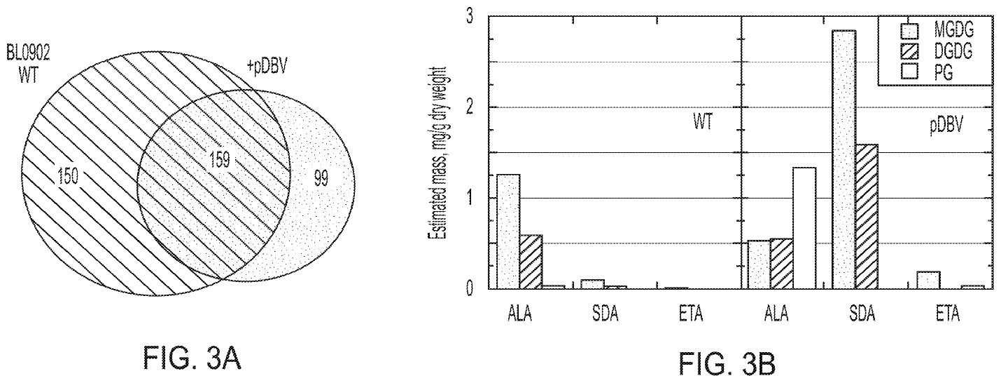

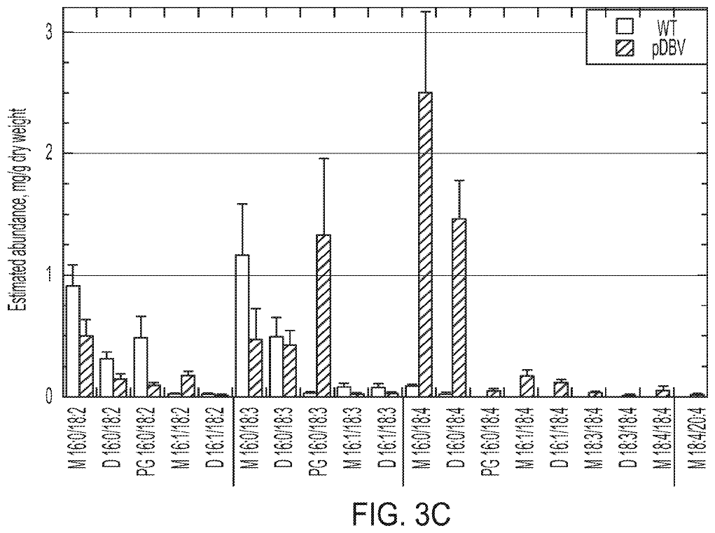

Lipidomics analysis was performed by liquid chromatography with tandem mass spectrometry (LC-MS/MS) using a high resolution Q Exactive HF Hybrid Quadrupole-Orbitrap mass spectrometer and was utilized to determine the molecular classes and species of all lipids, but especially those containing ALA-, SDA- and ETA. More than 300 lipid molecular species were identified in wild type BL0902 (309 total) and 258 species in the pDBV exconjugants (n=4 per group). FIG. 3 illustrates that there are 159 lipid molecular species shared by the wild type and pDBV exconjugants, while 99 lipid molecular species were unique to the exconjugants. These unique lipids included ALA-, SDA-, ETA-containing molecular species of MGDG and DGDG classes (FIG. 3C). Importantly, SDA is found almost entirely in the MGDG and DGDG species (Table 2, FIGS. 3B and 3C). In contrast, ALA is predominantly in MGDG and DGDG in WT, but is highly enriched in phosphatidylglycerol (PG, 56%) in the pDBV exconjugant and ETA is found predominantly in MGDG (84%) with none detected in DGDG (FIG. 3B).

The individual molecular species of MGDG, DGDG and PG are illustrated in FIG. 3C. Glycolipids contain 18C acyl groups such as 18:0, 18:1 (n-9), 18:2 (n-6), 18:3 (n-3 or n-6) at the sn-1 position and C16 acyl groups including 16:0, 16:1, 16:2, 16:3 at the sn-2 position of the molecule (29). In the case of MGDG, DGDG, and PG containing ALA, SDA or ETA, the most common fatty acid observed at the sn-2 acyl position is palmitic acid (16:0) (89-95% of the time). Interestingly, the pDBV exconjugants produced several novel and unexpected MGDG and DGDG molecular species containing 18C and/or 20C PUFAs in both (sn-1 and sn-2) positions, including SDA and ALA, SDA and SDA, or SDA and ETA (FIG. 3C).

A second experiment comparing BL0902 and 7002 samples with and without pDBV demonstrated similar molecular profiles, with ALA/ALA, ALA/SDA, ALA/ETA, SDA/SDA, and SDA/ETA “double omega-3” chains among the MGDG and DGDG molecular species, as well as the SDA/SDA molecular species in sulfoquinovosyldiacylglycerol (SQDG). There also were other rarer PUFAs induced by pDBV in both BL0902 and 7002, including 16:3, 16:4, 17:2, 17:4, 20:3, and 20:5 (Supplementary Data Excel Spreadsheet and Table 3). Another highly unusual fatty acid, 18:5, was present in MGDG and SQDG in the second experiment, and the SQDG, in particular, was induced upon addition of pDBV to both BL0902 and 7002.

Summary of Unusual Lipids and Fatty Acids Detected by Lipidomics (Mass Spectrometry) and Gas Chromatography-Flame Ionization Detection (GC-FID) with Fatty Acid Methyl Esters.

Listed below are species observed in the course of the two lipidomics experiments and the GC-FID analysis described above and in brief below. E1 refers to experiment 1 (Leptolynbya sp. strain BL0902+/−pDBV, n=4 each; also analyzed Spirulina lipids, not shown here) E2 refers to experiment 2 (Leptolynbya sp. strain BL0902 & Synechococcus sp. PCC7002+ or −pDBV, n=1 each)
Most are found in MGDG, often also in DGDG; no differences between BL0902 & 7002 species in E2 for presence/absence of species
MG=MGDG=monogalactosyldiacylglycerol; DG=DGDG=digalactosyldiacylglycerol; SQ=SQDG=sulfoquinovosyldiacylglycerol; PG=phosphatidylglycerol
Highlighted in BOLD if only in +pDBV, underlined if “induced” in +pDBV

### Fatty Acid Chains:

### 16:3, MG, E1, MG & DG E2

16:4, MG, E1 only
17:1, MG, E2 only
17:2, MG, DG and PG (both experiments, except not PG E2)
17:3, MG, DG and PG (both experiments, except not DG E2)
17:4, MG & DG, but MG only E2
17:5, with 23:2 and 23:3 in SQ only, E1 only
18:5, MG & SQ, E2 only (MG16:0/18:5 induced in 0902, opposite in 7002; SQ18:1/18:5 and SQ 18:2/18:5 only in +pDBV)
19:1, MG & DG, E1 only
19:2, MG & DG, E1 only
19:3, MG, both experiments

### 20:3, MG & DG E1, MG & PG E2

20:5, MG only, E2 only (both species)
18:4, 20:4, 20:5 were also found only in +pDBV

### Molecular Species (by Lipidomics):

### Expected Potent Anti-Inflammatory Molecules:

ALA=18:3 (omega-3), SDA=18:4 (omega-3), ETA=20:4 (omega-3 analogue of arachidonic acid)
ALA/ALA, MG & DG, mainly in E2
ALA/SDA, MG & DG, both experiments (trace in MG E1 & E2)
ALA/ETA, MG & DG, only E2
SDA/SDA, MG only in E1; MG, DG & SQ in E2
SDA/ETA, MG only in E1; MG & DG in E2
Also unusual: 17:5/23:2 or 17:5/23:3 in SQ only, E1 only

### Additional Molecules:

Experiment 2 only:

### 17:2/18:4, MG & DG

17:3/18:4, MG & DG (MG almost 0 in WT)
18:1/18:5, S_Q only
18:2/18:5, SQ only
Expt 1 (n=4 each), only seen in +pDBV

### M14:0/18:4 & D14:0/18:4

### M16:0/16:4

### M16:0/17:4 & D16:0/17:4

### PG16:0/20:4

### M17:0/18:4

### D18:3/18:4

### M18:4/18:4

### M18:4/20:4

This work was set out to determine the capacity of cyanobacteria strains to produce SDA as well as elongation and desaturation metabolites (ETA and EPA, respectively) complexed to potentially more bioavailable glycolipids (MGDG and DGDG). It was accomplished by conjugal transfer into three cyanobacteria of a three-gene plasmid, pDBV, which encodes the thylakoid membrane-promoting protein Vipp1 and two acyl-lipid desaturases (Δ6 and Δ15 desaturases) that occur naturally in cyanobacteria. The total yield of fatty acids increased three-fold in the pDBV exconjugants, and SDA levels which were at baseline in the wild type cyanobacteria rose to 26.6 mol % in Leptolyngbya BL0902 and 17.3 mol % in Synechococcus PCC 7002. Importantly, n-3 PUFAs and LC-PUFAs (ALA+SDA+ETA) comprised ˜40% of total fatty acids in engineered Leptolyngbya BL0902, and these were incorporated into MGDG and DGDG with a n-3 to n-6 PUFAs ratio of >50:1 (Table 1).

The rationale for the addition of vipp1 to the plasmid was to enhance thylakoid membranes and thus the content of MGDG and DGDG as well as the potential activities of “Group 4” acyl-lipid desaturases (DesC, DesA, DesB, and DesD). While Vipp1 is reported to promote thylakoid membrane biogenesis and maintenance in plants and cyanobacteria associated with enhanced photosynthetic machinery (30, 31), the effect of this gene on PUFA and LC-PUFA content has not been previously reported. Enhancement of SDA and ETA content even further in these cyanobacteria could take advantage of approaches reported previously by others, including growth at lower temperatures and higher light intensities and further engineering of the cyanobacteria to enhance their capacity to generate more precursor substrates (LA, GLA or ALA) necessary for SDA and ETA production. For example, Spirulina strains can contain ˜50% of their total fatty acids as LA and GLA (32), and other cyanobacterial strains have been engineered that contain 25-82% of their total fatty acids as LA and ALA (33-35).

Given SDA's stability relative to n-3 LC-PUFAs in food matrices and its capacity to be more efficiently (than ALA) converted to health-promoting EPA in humans, fish, and livestock, a great deal of effort has gone into finding natural systems and designing new engineered pathways that produce high quantities of SDA and high ratios of n-3 to n-6 PUFAs (36). SDA is seldom naturally found in cyanobacteria (37) unless at least one acyl-lipid desaturase is provided on a multicopy vector (35), and even then, the maximal content of 26.6% SDA reached in the current study is at least two-fold higher than previous engineered strains (33, 35). SDA-producing transgenic soybean oil contains 20-26% of the total fatty acids as SDA with n-3 to n-6 PUFA ratios of ˜1 or lower (18, 36, 38, 39), and seed oil from Echium plantagineum naturally contains ˜13% of the total fatty acids as SDA (15, 40, 41).

Lipidomics analysis identified complex lipids and individual molecular species containing newly-synthesized n-3 PUFAs and LC-PUFAs. Greater than 99% of SDA and ETA resided in MGDG and DGDG, with the majority at the sn-1 position and palmitic acid at the sn-2 position of the glycolipid backbone (FIG. 3B). Additionally, several highly unusual MGDG and DGDG molecular species containing SDA at both acyl positions or ALA:SDA, ALA:ETA, and SDA:ETA combinations were detected. ETA is a rare n-3 LC-PUFA in nature comprising ˜1% of fish oils. ETA is also found in triglycerides of transgenic seeds from Camelina and in New Zealand green-lipped mussel (Perna canaliculus) (42). ETA (also known as omega-3 arachidonic acid) an elongation product of SDA, is a structural analog of the n-6 arachidonic acid. Previous studies have demonstrated ETA's capacity to serve as a dual inhibitor of cyclooxygenases (COX1 and COX2) and lipoxygenases that can block the production of several classes of pro-inflammatory eicosanoids including leukotrienes, prostaglandins, and thromboxanes (43, 44). ETA has also been demonstrated to compete with arachidonic acid at the arachidonoyl-CoA synthetase step thereby preventing arachidonic acid uptake (42, 45). Importantly, lipid extracts from New Zealand green-lipped mussel also have been shown to have benefits in patients with atopic asthma (46). Future studies will determine whether molecular species of MGDG and DGDG such as ALA:ETA, SDA:ETA, and ETA:ETA have the potential to serve as bioavailable anti-inflammatory compounds.

Significant advantages of sourcing SDA and ETA from cyanobacteria include: (1) they require minimal nutritional demands, relying on photosynthesis and carbon dioxide rather than fermentable sugars (47); (2) they are able to serve directly as single ingredient feeds for aquaculture and livestock (simply by drying and being fed as flakes or pellets), therefore offering the possibility of less labor- and land-intensive cultivation of such feeds (20); (3) cyanobacteria such as Spirulina are currently used as food supplements because of their high protein content and digestibility (20); and (4) SDA and ETA formed in cyanobacteria, as shown here, are complexed to bioavailable, polar glycolipids. Human and rodent studies show that high doses of echium oil reduces circulating triglycerides (15, 48) and that >1 g/day of SDA from transgenic soybean oil is effective at raising tissue membrane levels of EPA and improving the omega-3 index in humans (erythrocyte EPA and DHA) (18, 49-51). SDA-enriched soybean oil fed to laying hens performs better than ALA at enriching eggs with n-3 LC-PUFAs and particularly EPA (52), and similar effects were obtained with meat from broiler chickens (53). Echium oil also enhances total n-3 PUFA levels, including EPA, in the milk of dairy cattle (54). However, there continue to be substantial barriers to the supplementation of SDA complexed to triglycerides (as found in seed and soybean oils), and this has limited the widespread use of SDA. Initial studies suggest that PUFAs or LC-PUFAs complexed to MGDG and DGDG may provide greater bioavailability than non-polar, triglyceride-containing oils and phospholipids found in krill oil (21).

This disclosure demonstrated that cyanobacteria, and especially Leptolyngbya BL0902, bioengineered with cyanobacterial lipid biosynthetic promoting genes, produce large quantities of SDA up to about 27% of total fatty acids. Importantly, both newly-synthesized SDA, ETA and EPA are found conjugated to galactose-containing glycerol backbones in what initial studies indicate is a more bioavailable polar lipid form than the neutral storage lipids like triacylglycerols of oils. Most importantly, a number of novel and potentially beneficial molecular species of MGDG and DGDG are formed in these cyanobacteria, which contain omega-3 fatty acid chains in both the sn-1 and sn-2 positions that may serve as potent, highly bioavailable, anti-inflammatory compounds. Omega-3 producing cyanobacteria as developed here are thus promising, sustainable sources of omega-3 PUFAs that could replace unstable fish oil products and fish meal as nutritional supplements for human, agricultural and aquacultural use. Further, the novel, anti-inflamatory “double omega-3” molecules produced by the engineered cyanobacteria are molecules not observed in nature, which could be utilized as highly bioavailable, anti-inflammatory pharmaceuticals in place of NSAIDs.

TABLE 2
Lipidomics analysis of molecular species in wild type (WT) and modified (+pDBV) Leptolyngbya sp. strain BL0902^{a}.

| Molecular | MGDG^{c} | | DGDG | | SQDG | | PG | |
|---|---|---|---|---|---|---|---|---|
| species^{b} | WT | +pDBV | WT | +pDBV | WT | +pDBV | WT | +pDBV |
| 14:0/16:0 | | | 0.005 ± 0.001 | 0.002 ± 0.001 | | | 0.001 ± 0.001 | 0.000 ± 0.000 |
| 14:0/18:3 | | | 0.013 ± 0.001 | 0.010 ± 0.003 | | | | |
| 14:0/18:4 | 0.000 ± 0.000 | 0.009 ± 0.002 | 0.000 ± 0.000 | 0.006 ± 0.002 | | | | |
| 16:0/16:0 | 0.066 ± 0.024 | 0.129 ± 0.038 | 0.043 ± 0.022 | 0.150 ± 0.111 | 0.008 ± 0.005 | 0.016 ± 0.015 | 0.037 ± 0.023 | 0.113 ± 0.037 |
| 16:0/16:1 | 0.161 ± 0.028 | 0.201 ± 0.080 | 0.045 ± 0.018 | 0.078 ± 0.024 | | | 0.077 ± 0.025 | 0.089 ± 0.028 |
| 16:0/16:2 | 0.021 ± 0.006 | 0.016 ± 0.005 | 0.011 ± 0.004 | 0.006 ± 0.001 | | | 0.004 ± 0.002 | 0.007 ± 0.003 |
| 16:0/16:3 | 0.001 ± 0.000 | 0.040 ± 0.018 | | | | | | |
| 16:0/16:4 | 0.000 ± 0.000 | 0.010 ± 0.003 | | | | | | |
| 16:0/17:0 | 0.002 ± 0.001 | 0.007 ± 0.002 | 0.002 ± 0.002 | 0.012 ± 0.005 | | | 0.000 ± 0.001 | 0.001 ± 0.002 |
| 16:0/17:1 | 0.040 ± 0.010 | 0.066 ± 0.020 | 0.009 ± 0.004 | 0.016 ± 0.003 | | | 0.013 ± 0.005 | 0.044 ± 0.020 |
| 16:0/17:2 | 0.028 ± 0.006 | 0.034 ± 0.012 | 0.011 ± 0.003 | 0.009 ± 0.002 | | | 0.005 ± 0.002 | 0.001 ± 0.002 |
| 16:0/17:3 | 0.020 ± 0.008 | 0.024 ± 0.004 | 0.010 ± 0.006 | 0.010 ± 0.006 | | | 0.000 ± 0.000 | 0.014 ± 0.005 |
| 16:0/17:4 | 0.000 ± 0.000 | 0.045 ± 0.014 | 0.000 ± 0.000 | 0.015 ± 0.003 | | | | |
| 16:0/18:0 | 0.008 ± 0.005 | 0.020 ± 0.019 | 0.007 ± 0.008 | 0.029 ± 0.011 | | | 0.002 ± 0.002 | 0.006 ± 0.002 |
| 16:0/18:1 | 0.410 ± 0.088 | 0.488 ± 0.180 | 0.087 ± 0.044 | 0.185 ± 0.057 | | | 0.275 ± 0.068 | 0.397 ± 0.176 |
| 16:0/18:2 | 0.908 ± 0.179 | 0.501 ± 0.132 | 0.311 ± 0.059 | 0.145 ± 0.039 | | | 0.484 ± 0.177 | 0.093 ± 0.022 |
| 16:0/18:3 | 1.166 ± 0.421 | 0.469 ± 0.254 | 0.491 ± 0.160 | 0.424 ± 0.118 | | | 0.027 ± 0.013 | 1.325 ± 0.636 |
| 16:0/18:4 | 0.090 ± 0.011 | 2.507 ± 0.665 | 0.022 ± 0.013 | 1.462 ± 0.321 | | | 0.001 ± 0.001 | 0.046 ± 0.021 |
| 16:0/19:1 | 0.007 ± 0.001 | 0.017 ± 0.006 | 0.002 ± 0.001 | 0.004 ± 0.001 | | | | |
| 16:0/19:2 | 0.006 ± 0.004 | 0.002 ± 0.001 | 0.003 ± 0.002 | 0.001 ± 0.000 | | | | |
| 16:0/19:3 | 0.002 ± 0.001 | 0.007 ± 0.001 | | | | | | |
| 16:0/20:0 | 0.000 ± 0.000 | 0.001 ± 0.001 | | | | | | |
| 16:0/20:2 | 0.004 ± 0.003 | 0.003 ± 0.001 | 0.003 ± 0.003 | 0.001 ± 0.000 | | | | |
| 16:0/20:3 | 0.012 ± 0.009 | 0.033 ± 0.010 | 0.005 ± 0.004 | 0.010 ± 0.003 | | | 0.001 ± 0.002 | 0.004 ± 0.001 |
| 16:0/20:4 | 0.006 ± 0.004 | 0.174 ± 0.065 | | | | | 0.000 ± 0.000 | 0.037 ± 0.021 |
| 16:0/20:5 | 0.077 ± 0.010 | 0.122 ± 0.070 | | | | | | |
| 16:1/16:1 | 0.005 ± 0.001 | 0.007 ± 0.002 | 0.005 ± 0.001 | 0.003 ± 0.001 | | | | |
| 16:1/16:2 | 0.001 ± 0.001 | 0.000 ± 0.000 | | | | | | |
| 16:1/17:3 | 0.001 ± 0.001 | 0.001 ± 0.000 | 0.001 ± 0.001 | 0.000 ± 0.000 | | | | |
| 16:1/18:2 | 0.022 ± 0.004 | 0.171 ± 0.036 | 0.019 ± 0.005 | 0.004 ± 0.001 | | | | |
| 16:1/18:3 | 0.080 ± 0.028 | 0.018 ± 0.011 | 0.076 ± 0.030 | 0.026 ± 0.009 | | | 0.000 ± 0.000 | 0.006 ± 0.001 |
| 16:1/18:4 | 0.003 ± 0.001 | 0.166 ± 0.051 | 0.002 ± 0.001 | 0.115 ± 0.027 | | | | |
| 17:0/18:3 | 0.001 ± 0.000 | 0.002 ± 0.001 | | | | | | |
| 17:0/18:4 | 0.001 ± 0.000 | 0.006 ± 0.004 | | | | | | |
| 17:1/18:3 | 0.001 ± 0.001 | 0.001 ± 0.001 | 0.001 ± 0.001 | 0.000 ± 0.000 | | | | |
| 17:5/23:2 | | | | | 1.089 ± 0.123 | 2.172 ± 0.449 | | |
| 17:5/23:3 | | | | | 0.005 ± 0.003 | 0.006 ± 0.003 | | |
| 18:0/18:1 | 0.001 ± 0.001 | 0.002 ± 0.001 | | | | | 0.001 ± 0.000 | 0.002 ± 0.001 |
| 18:1/18:2 | 0.005 ± 0.005 | 0.001 ± 0.001 | | | | | 0.001 ± 0.000 | 0.008 ± 0.003 |
| 18:1/18:3 | | | 0.002 ± 0.001 | 0.071 ± 0.027 | | | | |
| 18:2/18:2 | 0.009 ± 0.003 | 0.011 ± 0.004 | 0.003 ± 0.002 | 0.001 ± 0.001 | | | | |
| 18:2/18:3 | 0.005 ± 0.004 | 0.005 ± 0.004 | 0.005 ± 0.003 | 0.006 ± 0.005 | | | | |
| 18:3/18:3 | 0.003 ± 0.003 | 0.001 ± 0.001 | 0.002 ± 0.001 | 0.000 ± 0.000 | | | | |
| 18:3/18:4 | 0.000 ± 0.000 | 0.032 ± 0.015 | 0.000 ± 0.000 | 0.012 ± 0.004 | | | | |
| 18:4/18:4 | 0.000 ± 0.000 | 0.049 ± 0.037 | | | | | | |
| 18:4/20:4 | 0.000 ± 0.000 | 0.012 ± 0.009 | | | | | | |

^{a}Values reported are estimates of mg/g of total fatty acid based on (i) normalized peak areas from LC/MS, (ii) fraction of total peak area for each species in a sample, and (iii) known total fatty acid yield for that organism from GC-FID analysis. This treatment assumes that all species exhibit the same ionization efficiency. Shown aremean +/− standard deviation; data shown are for wild type (WT) Leptolyngbya BL0902 and the same strain with pDBV (n = 4 for each). Molecular species containing at least one acyl group with SDA (18:4), ETA (20:4) or EPA (20:5) are underlined. No entry means that the species was not observed in the WT or pDBV samples in that category.
^{b}Molecular species shown are for the two fatty acyl chains, giving carbon chain length and number of double bonds for each (e.g., 16:0 means 16 carbons long with 0 double bonds).
^{c}MGDG = monogalactosyldiacylglycerol; DGDG = digalactosyldiacylglycerol; SQDG = sulfoquinovosyldiacylglycerol; PG = phosphatidyl glycerol

TABLE 3
Molecular species of lipids by LC-MS/MS in wild type (WT)
and engineered (+pDBV) Leptolyngbya sp. strain BL0902
and Synechococcus sp. PCC 7002 (n = 1 each).^{a}

| Molecular | Cyano | MGDG | | DGDG | | SQDG | | PG | |
|---|---|---|---|---|---|---|---|---|---|
| species | strain | WT | +pDB | WT | +pDBV | WT | +pDBV | WT | +pDBV |
| 14:0/16:0 | BL0902 | 0.111 | 0.850 | 0.000 | 0.000 | | | | |
| | 7002 | 0.225 | 0.112 | 0.012 | 0.008 | | | | |
| 14:0/18:3 | BL0902 | 0.008 | 0.042 | 0.012 | 0.008 | | | 0.000 | 0.009 |
| | 7002 | 0.017 | 0.008 | 0.015 | 0.001 | | | 0.001 | 0.010 |
| 14:0/18:4 | BL0902 | 0.000 | 0.168 | 0.001 | 0.012 | | | | |
| | 7002 | 0.000 | 0.022 | 0.003 | 0.004 | | | | |
| 16:0/16:0 | BL0902 | 0.051 | 0.796 | 0.000 | 0.012 | 0.005 | 0.006 | 0.001 | 0.256 |
| | 7002 | 0.053 | 0.030 | 0.000 | 0.004 | 0.027 | 0.008 | 0.015 | 0.032 |
| 16:0/16:1 | BL0902 | 0.090 | 0.508 | 0.001 | 0.045 | | | 0.003 | 0.105 |
| | 7002 | 0.072 | 0.022 | 0.002 | 0.004 | | | 0.018 | 0.039 |
| 16:0/16:3 | BL0902 | 0.000 | 0.051 | | | | | | |
| | 7002 | 0.000 | 0.014 | | | | | | |
| 16:0/17:2 | BL0902 | 0.019 | 0.065 | | | | | 0.000 | 0.010 |
| | 7002 | 0.057 | 0.030 | | | | | 0.011 | 0.005 |
| 16:0/17:4 | BL0902 | 0.000 | 0.024 | | | | | | |
| | 7002 | 0.000 | 0.015 | | | | | | |
| 16:0/18:1 | BL0902 | 0.629 | 1.479 | 0.000 | 0.000 | | | 0.207 | 1.164 |
| | 7002 | 0.676 | 0.273 | 0.005 | 0.003 | | | 0.640 | 0.353 |
| 16:0/18:2 | BL0902 | 0.566 | 3.214 | 0.011 | 0.134 | | | 0.057 | 1.843 |
| | 7002 | 3.964 | 0.979 | 0.030 | 0.056 | | | 1.710 | 0.266 |
| 16:0/18:3 | BL0902 | 0.168 | 1.582 | 0.017 | 0.058 | 0.128 | 2.301 | | |
| | 7002 | 0.692 | 0.858 | 0.325 | 0.011 | 0.796 | 4.914 | | |
| 16:0/18:4 | BL0902 | 0.001 | 5.330 | 0.012 | 0.212 | 0.004 | 1.109 | 0.000 | 0.035 |
| | 7002 | 0.001 | 2.199 | 0.126 | 0.123 | 0.000 | 0.235 | 0.000 | 0.013 |
| 16:0/18:5 | BL0902 | 0.001 | 0.009 | | | | | | |
| | 7002 | 0.025 | 0.008 | | | | | | |
| 16:0/19:2 | BL0902 | 0.007 | 0.011 | | | | | | |
| | 7002 | 0.029 | 0.005 | | | | | | |
| 16:0/20:4 | BL0902 | 0.053 | 0.527 | 0.000 | 0.712 | | | 0.000 | 0.114 |
| | 7002 | 0.015 | 0.145 | 0.000 | 0.295 | | | 0.000 | 0.035 |
| 16:0/20:5 | BL0902 | | | 0.006 | 0.038 | 0.031 | 0.023 | | |
| | 7002 | | | 0.003 | 0.011 | 0.019 | 0.023 | | |
| 16:1/18:2 | BL0902 | 0.483 | 0.122 | 0.000 | 0.000 | | | 0.010 | 2.403 |
| | 7002 | 0.580 | 0.021 | 0.003 | 0.000 | | | 0.464 | 2.173 |
| 16:1/18:3 | BL0902 | 0.100 | 0.180 | 0.048 | 0.014 | 0.014 | 0.088 | 0.017 | 0.046 |
| | 7002 | 0.161 | 0.163 | 0.004 | 0.004 | 0.001 | 0.059 | 0.018 | 0.057 |
| 16:1/18:4 | BL0902 | 0.001 | 1.006 | 0.013 | 0.467 | | | | |
| | 7002 | 0.000 | 0.363 | 0.116 | 0.282 | | | | |
| 16:2/18:3 | BL0902 | | | 0.000 | 0.025 | | | | |
| | 7002 | | | 0.000 | 0.010 | | | | |
| 16:2/18:4 | BL0902 | 0.013 | 0.117 | 0.000 | 0.014 | | | | |
| | 7002 | 0.003 | 0.053 | 0.005 | 0.008 | | | | |
| 16:3/18:4 | BL0902 | 0.000 | 0.021 | 0.000 | 0.053 | | | | |
| | 7002 | 0.000 | 0.011 | 0.000 | 0.033 | | | | |
| 17:1/18:2 | BL0902 | 0.019 | 0.003 | | | | | | |
| | 7002 | 0.051 | 0.003 | | | | | | |
| 17:1/18:4 | BL0902 | 0.000 | 0.016 | | | | | | |
| | 7002 | 0.000 | 0.017 | | | | | | |
| 17:2/18:4 | BL0902 | 0.000 | 0.014 | 0.000 | 0.000 | | | | |
| | 7002 | 0.000 | 0.011 | 0.003 | 0.002 | | | | |
| 17:3/18:4 | BL0902 | 0.000 | 0.007 | 0.000 | 0.003 | | | | |
| | 7002 | 0.000 | 0.008 | 0.000 | 0.004 | | | | |
| 18:0/18:4 | BL0902 | 0.005 | 0.012 | | | | | | |
| | 7002 | 0.001 | 0.011 | | | | | | |
| 18:1/18:1 | BL0902 | 0.199 | 0.030 | 0.000 | 0.000 | 0.090 | 0.028 | 0.018 | 0.306 |
| | 7002 | 0.081 | 0.013 | 0.004 | 0.001 | 0.134 | 0.030 | 0.070 | 0.014 |
| 18:1/18:2 | BL0902 | 0.479 | 0.114 | 0.007 | 0.000 | | | 0.015 | 0.050 |
| | 7002 | 0.272 | 0.070 | 0.005 | 0.000 | | | 0.041 | 0.005 |
| 18:1/18:4 | BL0902 | 0.194 | 0.470 | | | | | | |
| | 7002 | 0.222 | 0.256 | | | | | | |
| 18:1/18:5 | BL0902 | | | | | 0.003 | 0.065 | | |
| | 7002 | | | | | 0.002 | 0.065 | | |
| 18:1/20:4 | BL0902 | 0.000 | 0.014 | | | | | | |
| | 7002 | 0.002 | 0.007 | | | | | | |
| 18:2/18:2 | BL0902 | 0.330 | 0.027 | 0.029 | 0.000 | | | 0.003 | 0.017 |
| | 7002 | 0.554 | 0.041 | 0.032 | 0.001 | | | 0.008 | 0.017 |
| 18:2/18:3 | BL0902 | 0.079 | 0.207 | 0.028 | 0.019 | | | | |
| | 7002 | 0.281 | 0.106 | 0.186 | 0.013 | | | | |
| 18:2/18:4 | BL0902 | 0.005 | 0.097 | 0.011 | 0.026 | | | | |
| | 7002 | 0.001 | 0.022 | 0.127 | 0.031 | | | | |
| 18:2/18:5 | BL0902 | | | | | 0.000 | 0.088 | | |
| | 7002 | | | | | 0.000 | 0.031 | | |
| 18:2/20:4 | BL0902 | 0.000 | 0.019 | | | | | | |
| | 7002 | 0.001 | 0.015 | | | | | | |
| 18:3/18:3 | BL0902 | 0.013 | 0.405 | 0.000 | 0.020 | | | 0.000 | 0.001 |
| | 7002 | 0.030 | 0.094 | 0.000 | 0.014 | | | 0.001 | 0.026 |
| 18:3/18:4 | BL0902 | 0.000 | 0.502 | 0.010 | 0.087 | | | | |
| | 7002 | 0.001 | 0.261 | 0.015 | 0.063 | | | | |
| 18:3/20:3 | BL0902 | 0.000 | 0.006 | | | | | | |
| | 7002 | 0.000 | 0.009 | | | | | | |
| 18:3/20:4 | BL0902 | 0.000 | 0.029 | 0.000 | 0.210 | | | | |
| | 7002 | 0.000 | 0.016 | 0.000 | 0.111 | | | | |
| 18:4/18:4 | BL0902 | 0.000 | 0.868 | 0.000 | 0.014 | 0.000 | 0.075 | | |
| | 7002 | 0.000 | 0.203 | 0.000 | 0.013 | 0.000 | 0.006 | | |
| 18:4/20:4 | BL0902 | 0.000 | 0.114 | 0.000 | 0.167 | | | | |
| | 7002 | 0.000 | 0.048 | 0.000 | 0.049 | | | | |
| 18:4/20:5 | 7002 | 0.002 | 0.014 | | | | | | |

^{a}Values reported are estimates of mg/g of total fatty acid based on (i) normalized peak areas from LC/MS, (ii) fraction of total peak area for each species in a sample, and (iii) known total fatty acid yield for that organism from GC-FID analysis. This treatment assumes that all species exhibit the same ionization efficiency. Shown are mean +/− standard deviation for wild type (WT) Leptolyngbya BL0902 without and with pDBV, and Synechococcus sp. PCC 7002 (n = 1 each). No entry means that the species was not observed in the WT or pDBV samples in that category.

### F. References

1. Mozaffarian D, Wu J H. Omega-3 fatty acids and cardiovascular disease: effects on risk factors, molecular pathways, and clinical events. Journal of the American College of Cardiology. 2011; 58(20):2047-67.
2. Campoy C, Escolano-Margarit M V, Anjos T, Szajewska H, Uauy R. Omega 3 fatty acids on child growth, visual acuity and neurodevelopment. The British journal of nutrition. 2012; 107 Suppl 2:S85-106.
3. Grosso G, Galvano F, Marventano S, Malaguarnera M, Bucolo C, Drago F, Caraci F. Omega-3 fatty acids and depression: scientific evidence and biological mechanisms. Oxidative medicine and cellular longevity. 2014; 2014:313570.
4. Grosso G, Pajak A, Marventano S, Castellano S, Galvano F, Bucolo C, Drago F, Caraci F. Role of omega-3 fatty acids in the treatment of depressive disorders: a comprehensive meta-analysis of randomized clinical trials. PloS one. 2014; 9(5):e96905.
5. Luchtman D W, Song C. Cognitive enhancement by omega-3 fatty acids from child-hood to old age: findings from animal and clinical studies. Neuropharmacology. 2013; 64:550-65.
6. Adarme-Vega T C, Thomas-Hall S R, Schenk P M. Towards sustainable sources for omega-3 fatty acids production. Current opinion in biotechnology. 2014; 26:14-8.
7. Finco A M O, Mamani L D G, Carvalho J C, de Melo Pereira G V, Thomaz-Soccol V, Soccol C R. Technological trends and market perspectives for production of microbial oils rich in omega-3. Critical reviews in biotechnology. 2017; 37(5):656-71.
8. Carmona-Antonanzas G, Tocher D R, Martinez-Rubio L, Leaver M J. Conservation of lipid metabolic gene transcriptional regulatory networks in fish and mammals. Gene. 2014; 534(1):1-9.
9. Leaver M J, Villeneuve L A, Obach A, Jensen L, Bron J E, Tocher D R, Taggart J B. Functional genomics reveals increases in cholesterol biosynthetic genes and highly unsaturated fatty acid biosynthesis after dietary substitution of fish oil with vegetable oils in Atlantic salmon (Salmo salar). BMC genomics. 2008; 9:299.
10. Bell J G, Tocher D R, Henderson R J, Dick J R, Crampton V O. Altered fatty acid compositions in atlantic salmon (Salmo salar) fed diets containing linseed and rapeseed oils can be partially restored by a subsequent fish oil finishing diet. The Journal of nutrition. 2003; 133(9):2793-801.
11. Monge-Ortiz R, Tomis-Vidal A, Rodriguez-Barreto D, Martinez-Llorens S, Perez J A, Jover-Cerdi M, Lorenzo A. Replacement of fish oil with vegetable oil blends in feeds for greater amberjack (Seriola dumerili) juveniles: Effect on growth performance, feed efficiency, tissue fatty acid composition and flesh nutritional value. Aquaculture Nutrition. 2018; 24(1):605-15.
12. Ma X, Jiang Z, Lai C. Significance of Increasing n-3 PUFA Content in Pork on Human Health. Critical reviews in food science and nutrition. 2016; 56(5):858-70.
13. Lee S A, Whenham N, Bedford M R. Review on docosahexaenoic acid in poultry and swine nutrition: Consequence of enriched animal products on performance and health characteristics. Animal nutrition. 2019; 5(1):11-21.
14. James M J, Ursin V M, Cleland L G. Metabolism of stearidonic acid in human subjects: comparison with the metabolism of other n-3 fatty acids. The American journal of clinical nutrition. 2003; 77(5):1140-5.
15. Surette M E, Edens M, Chilton F H, Tramposch K M. Dietary echium oil increases plasma and neutrophil long-chain (n-3) fatty acids and lowers serum triacylglycerols in hypertriglyceridemic humans. The Journal of nutrition. 2004; 134(6):1406-11.
16. Whelan J. Dietary stearidonic acid is a long chain (n-3) polyunsaturated fatty acid with potential health benefits. The Journal of nutrition. 2009; 139(1):5-10.
17. Harris W S. The omega-3 index as a risk factor for coronary heart disease. The American journal of clinical nutrition. 2008; 87(6):1997S-2002S.
18. Lemke S L, Vicini J L, Su H, Goldstein D A, Nemeth M A, Krul E S, Harris W S. Dietary intake of stearidonic acid-enriched soybean oil increases the omega-3 index: randomized, double-blind clinical study of efficacy and safety. The American journal of clinical nutrition. 2010; 92(4):766-75.
19. Taton A, Lis E, Adin D M, Dong G, Cookson S, Kay S A, Golden S S, Golden J W. Gene transfer in Leptolyngbya sp. strain BL0902, a cyanobacterium suitable for production of biomass and bioproducts. PloS one. 2012; 7(1):e30901.
20. Thajuddin N, Subramanian G. Cyanobacterial biodiversity and potential applications in biotechnology. Current Science. 2005; 89:47-57.
21. Kagan M L, West A L, Zante C, Calder P C. Acute appearance of fatty acids in human plasma—a comparative study between polar-lipid rich oil from the microalgae Nannochloropsis oculata and krill oil in healthy young males. Lipids in health and disease. 2013; 12:102.
22. Dyerberg J, Madsen P, Moller J M, Aardestrup I, Schmidt E B. Bioavailability of marine n-3 fatty acid formulations. Prostaglandins, leukotrienes, and essential fatty acids. 2010; 83(3):137-41.
23. Sugawara T, Miyazawa T. Separation and determination of glycolipids from edible plant sources by high-performance liquid chromatography and evaporative light-scattering detection. Lipids. 1999; 34(11):1231-7.
24. Andersson L, Carriere F, Lowe M E, Nilsson A, Verger R. Pancreatic lipase-related protein 2 but not classical pancreatic lipase hydrolyzes galactolipids. Biochim Biophys Acta. 1996; 1302(3):236-40.
25. Kagan M L, Levy A, Leikin-Frenkel A. Comparative study of tissue deposition of omega-3 fatty acids from polar-lipid rich oil of the microalgae Nannochloropsis oculata with krill oil in rats. Food & function. 2015; 6(1):186-92.
26. Los D A, Mironov K S. Modes of Fatty Acid desaturation in cyanobacteria: an update. Life. 2015; 5(1):554-67.
27. Heidrich J, Thurotte A, Schneider D. Specific interaction of IM30/Vipp1 with cyanobacterial and chloroplast membranes results in membrane remodeling and eventually in membrane fusion. Biochimica et biophysica acta Biomembranes. 2017; 1859(4):537-49.
28. Kobayashi K, Endo K, Wada H. Specific Distribution of Phosphatidylglycerol to Photosystem Complexes in the Thylakoid Membrane. Frontiers in plant science. 2017; 8:1991.
29. Murata N, Wada H. Acyl-lipid desaturases and their importance in the tolerance and acclimatization to cold of cyanobacteria. Biochem J. 1995; 308 (Pt 1):1-8.
30. Kroll D, Meierhoff K, Bechtold N, Kinoshita M, Westphal S, Vothknecht U C, Soll J, Westhoff P. VIPP1, a nuclear gene of Arabidopsis thaliana essential for thylakoid membrane formation. Proc Natl Acad Sci USA. 2001; 98(7):4238-42.
31. Bohuszewicz O, Liu J, Low H H. Membrane remodelling in bacteria. J Struct Biol. 2016; 196(1):3-14.
32. Cohen Z, Vonshak A, Richmond A. Fatty acid composition of Spirulina strains grown under various environmental conditions. Phytochemistry. 1987; 26(8):2255-8.
33. Chen G, Qu S, Wang Q, Bian F, Peng Z, Zhang Y, Ge H, Yu J, Xuan N, Bi Y, He Q. Transgenic expression of delta-6 and delta-15 fatty acid desaturases enhances omega-3 polyunsaturated fatty acid accumulation in Synechocystis sp. PCC6803. Biotechnology for biofuels. 2014; 7(1):32.
34. Santos-Merino M, Garcillan-Barcia M P, de la Cruz F. Engineering the fatty acid synthesis pathway in Synechococcus elongatus PCC 7942 improves omega-3 fatty acid production. Biotechnology for biofuels. 2018; 11:239.
35. Yoshino T, Kakunaka N, Liang Y, Ito Y, Maeda Y, Nomaguchi T, Matsunaga T, Tanaka T. Production of omega3 fatty acids in marine cyanobacterium Synechococcus sp. strain NKBG 15041c via genetic engineering. Appl Microbiol Biotechnol. 2017; 101(18):6899-905.
36. Hammond B G, Lemen J K, Ahmed G, Miller K D, Kirkpatrick J, Fleeman T. Safety assessment of SDA soybean oil: results of a 28-day gavage study and a 90-day/one generation reproduction feeding study in rats. Regulatory toxicology and pharmacology: RTP. 2008; 52(3):311-23.
37. Guedes A C, Amaro H M, Barbosa C R, Pereira R D, Malcata F X. Fatty acid composition of several wild microalgae and cyanobacteria, with a focus on eicosapentaenoic, docoshexaenoic and alpha-linlenic acids for eventual dietary uses. Food Research International. 2011; 44:2721-9.
38. Eckert H, La Vallee B, Schweiger B J, Kinney A J, Cahoon E B, Clemente T. Co-expression of the borage Delta 6 desaturase and the Arabidopsis Delta 15 desaturase results in high accumulation of stearidonic acid in the seeds of transgenic soybean. Planta. 2006; 224(5):1050-7.
39. Ruiz-Lopez N, Haslam R P, Venegas-Caleron M, Larson T R, Graham I A, Napier J A, Sayanova O. The synthesis and accumulation of stearidonic acid in transgenic plants: a novel source of ‘heart-healthy’ omega-3 fatty acids. Plant biotechnology journal. 2009; 7(7):704-16.
40. Coupland K. Stearidonic acid: A plant produced omega-3 PUFA and a potential alternative for marine oil fatty acids. Lipid Technology. 2008; 20(7):152-4.
41. Zhao X-R, Robert S, Singh S, Green A. Heterologous production of GLA and SDA by expression of an Echium plantagineum Δ6-desaturase gene. Plant Sci. 2006; 170(3):665-73.
42. Treschow A P, Hodges L D, Wright P F, Wynne P M, Kalafatis N, Macrides T A. Novel anti-inflammatory omega-3 PUFAs from the New Zealand green-lipped mussel, Perna canaliculus. Comp Biochem Physiol B Biochem Mol Biol. 2007; 147(4):645-56.
43. Grienke U, Silke J, Tasdemir D. Bioactive compounds from marine mussels and their effects on human health. Food chemistry. 2014; 142:48-60.
44. McPhee S, Hodges L D, Wright P F, Wynne P M, Kalafatis N, Harney D W, Macrides T A. Anti-cyclooxygenase effects of lipid extracts from the New Zealand green-lipped mussel, Perna canaliculus. Comp Biochem Physiol B Biochem Mol Biol. 2007; 146(3):346-56.
45. Wilson D B, Prescott S M, Majerus P W. Discovery of an arachidonoyl coenzyme A synthetase in human platelets. J Biol Chem. 1982; 257(7):3510-5.
46. Emelyanov A, Fedoseev G, Krasnoschekova O, Abulimity A, Trendeleva T, Barnes P J. Treatment of asthma with lipid extract of New Zealand green-lipped mussel: a randomised clinical trial. The European respiratory journal. 2002; 20(3):596-600.
47. Roulet J, Taton A, Golden J W, Arabolaza A, Burkart M D, Gramajo H. Development of a cyanobacterial heterologous polyketide production platform. Metabolic engineering. 2018; 49:94-104.
48. Forrest L M, Lough C M, Chung S, Boudyguina E Y, Gebre A K, Smith T L, Colvin P L, Parks J S. Echium oil reduces plasma triglycerides by increasing intravascular lipolysis in apoB100-only low density lipoprotein (LDL) receptor knockout mice. Nutrients. 2013; 5(7):2629-45.
49. Deckelbaum R J, Calder P C, Harris W S, Akoh C C, Maki K C, Whelan J, Banz W J, Kennedy E. Conclusions and recommendations from the symposium, Heart Healthy Omega-3s for Food: Stearidonic Acid (SDA) as a Sustainable Choice. The Journal of nutrition. 2012; 142(3):641S-3S.
50. Harris W S, Lemke S L, Hansen S N, Goldstein D A, DiRienzo M A, Su H, Nemeth M A, Taylor M L, Ahmed G, George C. Stearidonic acid-enriched soybean oil increased the omega-3 index, an emerging cardiovascular risk marker. Lipids. 2008; 43(9):805-11.
51. Krul E S, Lemke S L, Mukherjea R, Taylor M L, Goldstein D A, Su H, Liu P, Lawless A, Harris W S, Maki K C. Effects of duration of treatment and dosage of eicosapentaenoic acid and stearidonic acid on red blood cell eicosapentaenoic acid content. Prostaglandins, leukotrienes, and essential fatty acids. 2012; 86(1-2):51-9.
52. Elkin R G, Ying Y, Harvatine K J. Feeding laying hens stearidonic acid-enriched soybean oil, as compared to flaxseed oil, more efficiently enriches eggs with very long-chain n-3 polyunsaturated fatty acids. Journal of agricultural and food chemistry. 2015; 63(10):2789-97.
53. Elkin R G, Ying Y, Fan Y, Harvatine K J. Influence of feeding stearidonic acid (18:4n-3)-enriched soybean oil, as compared to conventional soybean oil, on tissue deposition of very long-chain omega-3 fatty acids in meat-type chickens. Animal Feed Science and Technology. 2016; 217:1-12.
54. Kitessa S M, Young P. Enriching milk fat with n-3 polyunsaturated fatty acids by supplementing grazing dairy cows with ruminally protected Echium oil. Animal Feed Science and Technology. 2011; 170(1-2):35-44.
55. Stevens S E, Patterson C O, Myers J. The production of hydrogen peroxide by blue-green algae: A survey. J Phycology. 1973; 9:427-30.
56. Metcalfe L D, Schmitz A A, Pelka J R. Rapid Preparation of Fatty Acid Esters from Lipids for Gas Chromatographic Analysis. Analytical Chemistry. 1966; 38(3):514-5.
57. Weaver K L, Ivester P, Seeds M C, Case L D, Arm J, Chilton F H. Effect of dietary fatty acids on inflammatory gene expression in healthy humans. JBiolChem. 2009; 284:15400-7.
58. Sergeant S, Hugenschmidt C E, Rudock M E, Ziegler J T, Ivester P, Ainsworth H C, Vaidya D, Case L D, Langefeld C D, Freedman B I, Bowden D W, Mathias R A, Chilton F H. Differences in arachidonic acid levels and fatty acid desaturase (FADS) gene variants in African Americans and European Americans with diabetes or the metabolic syndrome. British Journal of Nutrition. 2012; 107(4):547-55.

1. A lipid composition comprising lipids and an omega-3 fatty acid selected from the group consisting of α-linolenic acid (ALA), stearidonic acid (SDA), omega-3 arachidonic acid (omega-3 ETA), and eicosapentaenoic acid (EPA), wherein the lipids comprise a glycolipid, and wherein a fraction of the omega-3 fatty acid is conjugated to at least one of the sn-1 and sn-2 positions of a glycolipid head group selected from the group consisting of monogalactosyldiacylglycerol (MGDG), digalactosyldiacylglycerol (DGDG), and sulfoquinovosyldiacylglycerol (SQDG).

2. The lipid composition of claim 1, wherein the lipids further comprise a phosphoglycerolipid.

3. The lipid composition of claim 2, further comprising a second fraction of the omega-3 fatty acid that is conjugated to at least one of the sn-1 and sn-2 positions of the phosphatidylglycerol (PG).

4. The lipid composition of claim 3, wherein the omega-3 fatty acid is conjugated to both the sn-1 and sn-2 positions of the MGDG, the DGDG, the SGQG, or the PG.

5. The lipid composition of any one of claim 3, wherein the omega-3 fatty acid conjugated to both the sn-1 and sn-2 positions of the MGDG, the DGDG, the SGQG, or the PG is selected from the group consisting of: 16:3/18:4, 17:2/18:4, 17:3/18:4, 18:2/20:4, 18:3/20:3, 18:3/18:4, 18:3/20:4, 18:3/20:5, 18:4/18:4, 18:4/20:4, 18:4/20:5, and 20:4/20:5.

6. The lipid composition of any one of claim 2, wherein the omega-3 fatty acid conjugated to the sn-1 position is the same as the omega-3 fatty acid conjugated to the sn-2 position.

7. The lipid composition of any one of claim 2, wherein the omega-3 fatty acid conjugated to the sn-1 position is different from the omega-3 fatty acid conjugated to the sn-2 position.

8. The lipid composition of claim 1, wherein at least 50% of the SDA, the omega-3 ETA, the EPA or the ALA is in the fraction of the omega-3 fatty acid conjugated to the MGDG, the DGDG, or both.

9. The lipid composition of claim 8, wherein at least 60% of the SDA is in the fraction of the omega-3 fatty acid conjugated to the MGDG, the DGDG, or both.

10. The lipid composition of claim 8, wherein at least 60% of the omega-3 ETA is in the fraction of the omega-3 fatty acid conjugated to the MGDG, the DGDG, or both.

11. The lipid composition of claim 1, comprising at least about 10% SDA, at least about 1% omega-3 ETA, at least about 0.1% EPA, or at least about 20% ALA, by weight of total fatty acid content.

12. The lipid composition of claim 1, wherein a total amount of the SDA, the omega-3 ETA, EPA, and the ALA is at least about 20% by weight of total fatty acid content.

13. The lipid composition of claim 1, comprising between about 5% and about 40% ALA; between about 10% and about 30% SDA; and between about 1% and about 10% omega-3 ETA.

14. The lipid composition of claim 1, wherein the lipid composition is produced from a modified cyanobacterium.

15. The lipid composition of claim 14, wherein the modified cyanobacterium is a species of Anabaena, Leptolyngbya, Lyngbya, Nostoc, Phormidium, Spirulina, Synechococcus, or Synechocystis.

16. The lipid composition of claim 14, wherein the modified cyanobacterium is Anabaena sp. PCC7120, Synechococcus sp. PCC7002, or Leptolyngbya sp. strain BL0902.

17. The lipid composition of claim 1, wherein the lipid composition is prepared in an administrable form selected from the group consisting of a pharmaceutical formulation, a nutritional formulation, a feed formulation, a dietary supplement, a medical food, a functional food, a beverage product, and a combination thereof.

18. A feed for use in aquaculture comprising the lipid composition of claim 1.

19. A food or drink additive comprising the lipid composition of claim 1.

20. A method of producing the lipid composition of claim 1, comprising:
culturing a modified microorganism comprising at least one exogenous gene encoding a desaturase in a culture medium under conditions in which the at least one exogenous gene encoding the desaturase is expressed; and
enriching the cultured modified microorganism from the culture medium,
wherein the cultured modified microorganism produces a greater amount of the lipids than does a culture comprising a control microorganism identical in all respects except that it does not include the at least one exogenous gene encoding the desaturase.

21. The method of claim 20, comprising extracting the lipids and the omega-3 fatty acid from biomass of the cultured modified microorganism.

22. The method of claim 20, wherein the modified microorganism further comprises an exogenous gene encoding thylakoid-promoting protein Vipp1.

23. The method of claim 20, wherein the at least one exogenous gene encoding the desaturase comprises a first desaturase gene encoding a Δ6 desaturase and a second desaturase gene encoding a Δ15 desaturase.

24. The method of claim 20, wherein the desaturase comprises a polypeptide sequence having at least about 75% identity to SEQ ID NO: 6 or 7 or comprising SEQ ID NO: 6 or 7.

25. The method of claim 22, wherein the Vipp1 comprises a polypeptide sequence having at least about 75% identity to SEQ ID NO: 5 or comprising SEQ ID NO: 5.

26. A pharmaceutical composition comprising the lipid composition of claim 1 and optionally a pharmaceutically acceptable carrier.

27. A method for preventing or treating omega-3 fatty acid deficiency in a subject, comprising administering an effective amount of the lipid composition of claim 1 to the subject in need thereof.

28. The method of claim 27, wherein the subject is a mammal.

29. The method of claim 27, wherein the subject is human.

30. The method of claim 29, wherein the human has a cardiovascular or inflammatory disease or condition, or a brain/psychological disorder.

31. A method of inhibiting a cyclooxygenase or a lipoxygenase in a subject, comprising administering an effective amount of the lipid composition of claim 1 to the subject in need thereof.

32. A method of inhibiting uptake of proinflammatory PUFAs such as arachidonic acid into phospholipids utilizing fatty acyl CoA synthetase or transferase activities or CoA-independent transacylase activity in a subject, comprising administering an effective amount of the lipid composition of claim 1 to a subject in need thereof.
